SEQUENCE LISTING (109 sequences)

SEQ ID NO: 1 - protein, 5 aa, Artificial Sequence
  Synthetic heavy chain CDR1 of AbF46
DYYMS

SEQ ID NO: 2 - protein, 19 aa, Artificial Sequence
  Synthetic heavy chain CDR2 of AbF46
FIRNKANGYTTEYSASVKG

SEQ ID NO: 3 - protein, 6 aa, Artificial Sequence
  Synthetic heavy chain CDR3 of AbF46
DNWFAY

SEQ ID NO: 4 - protein, 6 aa, Artificial Sequence
  Synthetic heavy chain CDR1 of c-Met antibody
  VARIANT (1): Xaa is Pro or Ser or absent
  VARIANT (2): Xaa is Glu or Asp
XXYYMS

SEQ ID NO: 5 - protein, 8 aa, Artificial Sequence
  Synthetic heavy chain CDR2 of c-Met antibody
  VARIANT (3): Xaa is Asn or Lys
  VARIANT (4): Xaa is Ala or Val
  VARIANT (7): X is Asn or Thr
RNXXNGXT

SEQ ID NO: 6 - protein, 6 aa, Artificial Sequence
  Synthetic heavy chain CDR3 of c-Met antibody
  VARIANT (5): Xaa is Ser or Thr
DNWLXY

SEQ ID NO: 7 - protein, 17 aa, Artificial Sequence
  Synthetic light chain CDR1 of c-Met antibody
  VARIANT (4): Xaa is His, Arg, Gln or Lys
  VARIANT (9): Xaa is Ser or Trp
  VARIANT (12): Xaa is His or Gln
  VARIANT (13): Xaa is Lys or Asn
KSSXSLLAXGNXXNYLA

SEQ ID NO: 8 - protein, 7 aa, Artificial Sequence
  Synthetic light chain CDR2 of c-Met antibody
  VARIANT (2): Xaa is Ala or Gly
  VARIANT (4): Xaa is Thr or Lys
  VARIANT (7): Xaa is Ser or Pro
WXSXRVX

SEQ ID NO: 9 - protein, 9 aa, Artificial Sequence
  Synthetic light chain CDR3 of c-Met antibody
  VARIANT (1): Xaa is Gly, Ala or Gln
  VARIANT (6): Xaa is Arg, His, Ser, Ala, Gly or Lys
  VARIANT (8): Xaa is Leu, Tyr, Phe or Met
XQSYSXPXT

SEQ ID NO: 10 - protein, 17 aa, Artificial Sequence
  Synthetic light chain CDR1 of AbF46
KSSQSLLASGNQNNYLA

SEQ ID NO: 11 - protein, 7 aa, Artificial Sequence
  Synthetic light chain CDR2 of AbF46
WASTRVS

SEQ ID NO: 12 - protein, 9 aa, Artificial Sequence
  Synthetic light chain CDR3 of AbF46
QQSYSAPLT

SEQ ID NO: 13 - protein, 9 aa, Artificial Sequence
  Synthetic CDR-L3 derived from L3-1 clone
QQSYSRPYT

SEQ ID NO: 14 - protein, 9 aa, Artificial Sequence
  Synthetic CDR-L3 derived from L3-2 clone
GQSYSRPLT

SEQ ID NO: 15 - protein, 9 aa, Artificial Sequence
  Synthetic CDR-L3 derived from L3-3 clone
AQSYSHPFS

SEQ ID NO: 16 - protein, 9 aa, Artificial Sequence
  Synthetic CDR-L3 derived from L3-5 clone
QQSYSRPFT

SEQ ID NO: 17 - protein, 117 aa, Artificial Sequence
  Synthetic heavy chain variable region of anti c-Met humanized antibody(huAbF46-H4)
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSS

SEQ ID NO: 18 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPYTFGQGTKVEIKR

SEQ ID NO: 19 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCGQSYSRPLTFGQGTKVEIKR

SEQ ID NO: 20 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCAQSYSHPFSFGQGTKVEIKR

SEQ ID NO: 21 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPFTFGQGTKVEIKR

SEQ ID NO: 22 - protein, 6 aa, Artificial Sequence
  Synthetic CDR-H1 derived from H11-4 clone
PEYYMS

SEQ ID NO: 23 - protein, 6 aa, Artificial Sequence
  Synthetic CDR-H1 derived from YC151 clone
PDYYMS

SEQ ID NO: 24 - protein, 6 aa, Artificial Sequence
  Synthetic CDR-H1 derived from YC193 clone
SDYYMS

SEQ ID NO: 25 - protein, 8 aa, Artificial Sequence
  Synthetic CDR-H2 derived from YC244 clone
RNNANGNT

SEQ ID NO: 26 - protein, 8 aa, Artificial Sequence
  Synthetic CDR-H2 derived from YC321 clone
RNKVNGYT

SEQ ID NO: 27 - protein, 6 aa, Artificial Sequence
  Synthetic CDR-H3 derived from YC354 clone
DNWLSY

SEQ ID NO: 28 - protein, 6 aa, Artificial Sequence
  Synthetic CDR-H3 derived from YC374 clone
DNWLTY

SEQ ID NO: 29 - protein, 17 aa, Artificial Sequence
  Synthetic CDR-L1 derived from L1-1 clone
KSSHSLLASGNQNNYLA

SEQ ID NO: 30 - protein, 17 aa, Artificial Sequence
  Synthetic CDR-L1 derived from L1-3 clone
KSSRSLLSSGNHKNYLA

SEQ ID NO: 31 - protein, 17 aa, Artificial Sequence
  Synthetic CDR-L1 derived from L1-4 clone
KSSKSLLASGNQNNYLA

SEQ ID NO: 32 - protein, 17 aa, Artificial Sequence
  Synthetic CDR-L1 derived from L1-12 clone
KSSRSLLASGNQNNYLA

SEQ ID NO: 33 - protein, 17 aa, Artificial Sequence
  Synthetic CDR-L1 derived from L1-22 clone
KSSHSLLASGNQNNYLA

SEQ ID NO: 34 - protein, 7 aa, Artificial Sequence
  Synthetic CDR-L2 derived from L2-9 clone
WASKRVS

SEQ ID NO: 35 - protein, 7 aa, Artificial Sequence
  Synthetic CDR-L2 derived from L2-12 clone
WGSTRVS

SEQ ID NO: 36 - protein, 7 aa, Artificial Sequence
  Synthetic CDR-L2 derived from L2-16 clone
WGSTRVP

SEQ ID NO: 37 - protein, 9 aa, Artificial Sequence
  Synthetic CDR-L3 derived from L3-32 clone
QQSYSKPFT

SEQ ID NO: 38 - DNA, 1416 bp, Artificial Sequence
  Synthetic nucleotide sequence of heavy chain of chAbF46
  misc_feature (1)..(6): EcoRI restriction site
  misc_feature (7)..(66): signal sequence
  misc_feature (67)..(417): VH - heavy chain variable region
  misc_feature (418)..(423): NdeI restriction site
  misc_feature (418)..(1407): CH - heavy chain constant region
  misc_feature (1408)..(1410): TGA - stop sodon
  misc_feature (1411)..(1416): XhoI restriction site
gaattcgccgccaccatggaatggagctgggtttttctcgtaacacttttaaatggtatccagtgtgaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaagcatcctctatcttcaaatggacaccctgagagctgaggacagtgccacttattactgtgcaagagataactggtttgcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 39 - DNA, 759 bp, Artificial Sequence
  Synthetic nucleotide sequence of light chain of chAbF46
  misc_difference (1)..(6): EcoRI restriction site
  misc_difference (7)..(90): signal sequence
  misc_difference (91)..(432): VL - light chain variable region
  misc_difference (430)..(435): BsiWI restriction site
  misc_difference (433)..(750): CL - light chain constant region
  misc_difference (751)..(753): stop codon
  misc_difference (754)..(759): XhoI restriction site
gaattcactagtgattaattcgccgccaccatggattcacaggcccaggtcctcatgttgctgctgctatcggtatctggtacctgtggagacattttgatgacccagtctccatcctccctgactgtgtcagcaggagagaaggtcactatgagctgcaagtccagtcagagtcttttagctagtggcaaccaaaataactacttggcctggcaccagcagaaaccaggacgatctcctaaaatgctgataatttgggcatccactagggtatctggagtccctgatcgcttcataggcagtggatctgggacggatttcactctgaccatcaacagtgtgcaggctgaagatctggctgtttattactgtcagcagtcctacagcgctccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 40 - protein, 447 aa, Artificial Sequence
  Synthetic amino acid sequence of H1-heavy
EVQLVESGGGLVQPGGSLRLSCAASGFTFTDYYMSWVRQAPGKGLEWLGFIRNKANGYTTEYSASVKGRFTISRDNSKNSLYLQMNSLKTEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 447 aa, Artificial Sequence
  Synthetic amino acid sequence of H3-heavy
EVQLVESGGGLVQPGGSLRLSCAASGFTFTDYYMSWVRQAPGKGLEWLGFIRNKANGYTTEYSASVKGRFTISRDNSKNSLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 447 aa, Artificial Sequence
  Synthetic amino acid sequence of H4-heavy
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 220 aa, Artificial Sequence
  Synthetic amino acid sequence of H1-light
DIVMTQSPDSLAVSLGERATINCKSSQSLLASGNQNNYLAWHQQKPGQPPKMLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 220 aa, Artificial Sequence
  Synthetic amino acid sequence of H2-light
DIVMTQTPLSLPVTPGEPASISCKSSQSLLASGNQNNYLAWHLQKPGQSPQMLIIWASTRVSGVPDRFSGSGSGTDFTLKISRVEAEDVGVYYCQQSYSAPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 220 aa, Artificial Sequence
  Synthetic amino acid sequence of H3-light
DIVMTQSPDSLAVSLGERATINCKSSQSLLASGNQNNYLAWYQQKPGQPPKLLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 219 aa, Artificial Sequence
  Synthetic amino acid sequence of H4-light
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSAPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 47 - DNA, 1350 bp, Artificial Sequence
  Synthetic nucleotide sequence of H1-heavy
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgaaaaccgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 48 - DNA, 1350 bp, Artificial Sequence
  Synthetic nucleotide sequence of H3-heavy
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgcgtgctgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 49 - DNA, 1350 bp, Artificial Sequence
  Synthetic nucleotide sequence of H4-heavy
gaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 50 - DNA, 669 bp, Artificial Sequence
  Synthetic nucleotide sequence of H1-light
gacatcgtgatgacccagtctccagactccctggctgtgtctctgggcgagagggccaccatcaactgcaagtccagccagagtcttttagctagcggcaaccaaaataactacttagcttggcaccagcagaaaccaggacagcctcctaagatgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 51 - DNA, 669 bp, Artificial Sequence
  Synthetic nucleotide sequence of H2-light
gatattgtgatgacccagactccactctccctgcccgtcacccctggagagccggcctccatctcctgcaagtccagtcagagtcttttagctagtggcaaccaaaataactacttggcctggcacctgcagaagccagggcagtctccacagatgctgatcatttgggcatccactagggtatctggagtcccagacaggttcagtggcagtgggtcaggcactgatttcacactgaaaatcagcagggtggaggctgaggatgttggagtttattactgccagcagtcctacagcgctccgctcacgttcggacagggtaccaagctggagctcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 52 - DNA, 669 bp, Artificial Sequence
  Synthetic nucleotide sequence of H3-light
gacatcgtgatgacccagtctccagactccctggctgtgtctctgggcgagagggccaccatcaactgcaagtccagccagagtcttttagctagcggcaaccaaaataactacttagcttggtaccagcagaaaccaggacagcctcctaagctgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 53 - DNA, 669 bp, Artificial Sequence
  Synthetic nucleotide sequence of H4-light
gatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagcgctccgctcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 54 - protein, 23 aa, Artificial Sequence
  Synthetic linker between VH and VL
GLGGLGGGGSGGGGSGGSSGVGS

SEQ ID NO: 55 - DNA, 1088 bp, Artificial Sequence
  Synthetic polynucleotide encoding scFv of huAbF46 antibody
gctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaac

SEQ ID NO: 56 - DNA, 5597 bp, Artificial Sequence
  Synthetic expression vector including polynucleotide encoding scFv of huAbF46 antibody
  misc_difference (573)..(578): NheI restriction site
  misc_difference (588)..(938): huAbF46 VH
  misc_difference (939)..(1007): linker
  misc_difference (1008)..(1349): huAbF46 VL
  misc_difference (1350)..(1355): EcoRI restriction site
  misc_difference (1356)..(1397): V5 epitope
  misc_difference (1398)..(1442): (G4S)3 linker
  misc_difference (1443)..(1649): Aga2
  misc_difference (1650)..(1652): TGA(stop codon)
  misc_difference (1653)..(1660): PmeI restriction site
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagctaattctacttcatacattttcaattaagatgcagttacttcgctgtttttcaatattttctgttattgctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaacccgctgatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaattgggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccgtgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaatttcttgaagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctggctagt

SEQ ID NO: 57 - protein, 13 aa, Artificial Sequence
  Synthetic U6-HC7 hinge
EPKSCDCHCPPCP

SEQ ID NO: 58 - DNA, 435 bp, Artificial Sequence
  Synthetic polynucleotide encoding CDR-L3 derived from L3-1 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacg

SEQ ID NO: 59 - DNA, 435 bp, Artificial Sequence
  Synthetic polynucleotide encoding CDR-L3 derived from L3-2 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgggcagtcctacagccgtccgctcacgttcggacagggtaccaaggtggagatcaaacgtacg

SEQ ID NO: 60 - DNA, 435 bp, Artificial Sequence
  Synthetic polynucleotide encoding CDR-L3 derived from L3-3 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgcacagtcctacagccatccgttctctttcggacagggtaccaaggtggagatcaaacgtacg

SEQ ID NO: 61 - DNA, 435 bp, Artificial Sequence
  Synthetic polynucleotide encoding CDR-L3 derived from L3-5 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtttacgttcggacagggtaccaaggtggagatcaaacgtacg

SEQ ID NO: 62 - protein, 462 aa, Artificial Sequence
  Synthetic polypeptide consisting of heavy chain of huAbF46-H4-A1, U6-HC7 hinge and constant region of human IgG1
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1410 bp, Artificial Sequence
  Synthetic polynucleotide encoding polypeptide consisting of heavy chain of huAbF46-H4-A1, U6-HC7 hinge and constant region of human IgG1
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaaagctgcgattgccactgtcctccatgtccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 64 - protein, 461 aa, Artificial Sequence
  Synthetic polypeptide consisting of heavy chain of huAbF46-H4-A1, human IgG2 hinge and constant region of human IgG1
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1407 bp, Artificial Sequence
  Synthetic polynucleotide encoding polypeptide consisting of heavy chain of huAbF46-H4-A1, human IgG2 hinge and constant region of human IgG1
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagaggaagtgctgtgtggagtgccccccctgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 66 - protein, 460 aa, Artificial Sequence
  Synthetic polypeptide consisting of heavy chain of huAbF46-H4-A1, human IgG2 hinge and constant region of human IgG2
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - DNA, 1404 bp, Artificial Sequence
  Synthetic polynucleotide encoding polypeptide consisting of heavy chain of huAbF46-H4-A1, human IgG2 hinge and constant region of human IgG2
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 68 - protein, 240 aa, Artificial Sequence [sequence omitted]

SEQ ID NO: 69 - DNA, 758 bp, Artificial Sequence
  Synthetic polynucleotide encoding polypeptide consisting of light chain of huAbF46-H4-A1(H36Y) and human kappa constant region
aattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggtaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 70 - protein, 240 aa, Artificial Sequence
  Synthetic polypeptide consisting of light chain of huAbF46-H4-A1 and human kappa constant region
MDSQAQVLMLLLLSVSGTCGDIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 19 aa, Artificial Sequence
  Synthetic epitope in SEMA domain of c-Met
FSPQIEEPSQCPDCVVSAL

SEQ ID NO: 72 - protein, 10 aa, Artificial Sequence
  Synthetic epitope in SEMA domain of c-Met
PQIEEPSQCP

SEQ ID NO: 73 - protein, 5 aa, Artificial Sequence
  Synthetic epitope in SEMA domain of c-Met
EEPSQ

SEQ ID NO: 74 - protein, 117 aa, Artificial Sequence
  Synthetic heavy chain variable region of anti-c-Met antibody (AbF46 or huAbF46-H1)
EVQLVESGGGLVQPGGSLRLSCAASGFTFTDYYMSWVRQAPGKGLEWLGFIRNKANGYTTEYSASVKGRFTISRDNSKNSLYLQMNSLKTEDTAVYYCARDNWFAYWGQGTLVTVSS

SEQ ID NO: 75 - protein, 114 aa, Artificial Sequence
  Synthetic light chain variable region of anti-c-Met antibody (AbF46 or huAbF46-H1)
DIVMTQSPDSLAVSLGERATINCKSSQSLLASGNQNNYLAWHQQKPGQPPKMLIIWASTRVSGVPDRFSGSGSGTDFTLTISSLQAEDVAVYYCQQSYSAPLTFGGGTKVEIKR

SEQ ID NO: 76 - DNA, 1416 bp, Artificial Sequence; identical to SEQ ID NO: 38
  Synthetic nucleotide sequence of heavy chain of nti-c-Met antibody (AbF46 or huAbF46-H1)
  misc_feature (1)..(6): EcoRI restriction site
  misc_feature (7)..(66): signal sequence
  misc_feature (67)..(417): VH - heavy chain variable region
  misc_feature (418)..(423): NdeI restriction site
  misc_feature (418)..(1407): CH - heavy chain constant region
  misc_feature (1408)..(1410): TGA - stop sodon
  misc_feature (1411)..(1416): XhoI restriction site

SEQ ID NO: 77 - DNA, 759 bp, Artificial Sequence; identical to SEQ ID NO: 39
  Synthetic nucleotide sequence of light chain of anti-c-Met antibody (AbF46 or huAbF46-H1)
  misc_difference (1)..(6): EcoRI restriction site
  misc_difference (7)..(90): signal sequence
  misc_difference (91)..(432): VL - light chain variable region
  misc_difference (430)..(435): BsiWI restriction site
  misc_difference (433)..(750): CL - light chain constant region
  misc_difference (751)..(753): stop codon
  misc_difference (754)..(759): XhoI restriction site

SEQ ID NO: 78 - DNA, 4170 bp, Artificial Sequence
  Synthetic polynucleotide encoding c-Met protein
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 79 - protein, 444 aa, Artificial Sequence
  Synthetic SEMA domain of c-Met
LHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNG

SEQ ID NO: 80 - protein, 451 aa, Artificial Sequence
  Synthetic PSI-IPT domain of c-Met
YTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTG

SEQ ID NO: 81 - protein, 313 aa, Artificial Sequence
  Synthetic TyrKc domain of c-Met
VHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 82 - DNA, 1332 bp, Artificial Sequence
  Synthetic polynucleotide encoding SEMA domain of c-Met
ctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggc

SEQ ID NO: 83 - DNA, 1299 bp, Artificial Sequence
  Synthetic polynucleotide encoding PSI-IPT domain of c-Met
tacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacagga

SEQ ID NO: 84 - DNA, 939 bp, Artificial Sequence
  Synthetic polynucleotide encoding TyrKc domain of c-Met
gtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 85 - protein, 13 aa, Artificial Sequence
  Synthetic heavy chain CDR3 of anti-c-Met antibody
DNWFAYWGQGTLV

SEQ ID NO: 86 - protein, 10 aa, Artificial Sequence
  Synthetic light chain CDR3 of anti-c-Met antibody
LTFGAGTKLE

SEQ ID NO: 87 - protein, 117 aa, Artificial Sequence
  Synthetic heavy chain variable region of monoclonal antibody AbF46
EVKLVESGGGLVQPGGSLRLSCATSGFTFTDYYMSWVRQPPGKALEWLGFIRNKANGYTTEYSASVKGRFTISRDNSQSILYLQMDTLRAEDSATYYCARDNWFAYWGQGTLVTVSA

SEQ ID NO: 88 - protein, 114 aa, Artificial Sequence
  Synthetic light chain variable region of anti-c-Met antibody
DILMTQSPSSLTVSAGEKVTMSCKSSQSLLASGNQNNYLAWHQQKPGRSPKMLIIWASTRVSGVPDRFIGSGSGTDFTLTINSVQAEDLAVYYCQQSYSAPLTFGAGTKLELKR

SEQ ID NO: 89 - protein, 17 aa, Artificial Sequence
  Synthetic light chain CDR3 of anti-c-Met antibody
QQSYSAPLTFGAGTKLE

SEQ ID NO: 90 - protein, 117 aa, Artificial Sequence
  Synthetic heavy chain variable region of AT-VH1
EVKLVESGGGLVQPGGSLRLSCATSGFTFTDYYMSWVRQPPGKGLEWLGFIRNKANGYTTEYSASVKGRFTISRDNSKSTLYLQMNSLRAEDSATYYCARDNWFAYWGQGTLVTVSS

SEQ ID NO: 91 - protein, 117 aa, Artificial Sequence
  Synthetic heavy chain variable region of AT-VH2
EVKLVESGGGLVQPGGSLRLSCATSGFTFTDYYMSWVRQPPGKGLEWLGFIRNKANGYTTEYSASVKGRFTISRDNSKSTLYLQMNSLRAEDTATYYCARDNWFAYWGQGTLVTVSS

SEQ ID NO: 92 - protein, 117 aa, Artificial Sequence
  Synthetic heavy chain variable region of AT-VH3
EVQLVESGGGLVQPGGSLRLSCATSGFTFTDYYMSWVRQPPGKGLEWLGFIRNKANGYTTEYSASVKGRFTISRDNSKSTLYLQMNSLRAEDTATYYCARDNWFAYWGQGTLVTVSS

SEQ ID NO: 93 - protein, 117 aa, Artificial Sequence
  Synthetic heavy chain variable region of AT-VH4
EVQLVESGGGLVQPGGSLRLSCATSGFTFTDYYMSWVRQPPGKGLEWLGFIRNKANGYTTEYSASVKGRFTISRDNSKNTLYLQMNSLRAEDTATYYCARDNWFAYWGQGTLVTVSS

SEQ ID NO: 94 - protein, 117 aa, Artificial Sequence
  Synthetic heavy chain variable region of AT-VH5
EVQLVESGGGLVQPGGSLRLSCATSGFTFTDYYMSWVRQPPGKGLEWLGFIRNKANGYTTEYSASVKGRFTISRDNSKNTLYLQMNSLRAEDTAVYYCARDNWFAYWGQGTLVTVSS

SEQ ID NO: 95 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSAPLTFGQGTKVEIKR

SEQ ID NO: 96 - protein, 113 aa, Artificial Sequence
  Synthetic light chain variable region of AT-Vk1
DILMTQSPSSLTASVGDRVTMTCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 97 - protein, 113 aa, Artificial Sequence
  Synthetic light chain variable region of AT-Vk2
DILMTQSPSSLSASVGDRVTITCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 98 - protein, 113 aa, Artificial Sequence
  Synthetic light chain variable region of AT-Vk3
DIQMTQSPSSLSASVGDRVTITCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 99 - protein, 113 aa, Artificial Sequence
  Synthetic light chain variable region of AT-Vk4
DIQMTQSPSSLSASVGDRVTITCKSSQSLLASGNQNNYLAWHQQKPGKAPKMLIIWASTRVSGVPDRFSGSGSGTDFTLTISSLQAEDVAVYYCQQSYSAPLTFGQGTKLEIK

SEQ ID NO: 100 - protein, 13 aa, Artificial Sequence
  Synthetic modified hinge region(U7-HC6)
EPSCDKHCCPPCP

SEQ ID NO: 101 - protein, 13 aa, Artificial Sequence
  Synthetic modified hinge region(U6-HC7)
EPKSCDCHCPPCP

SEQ ID NO: 102 - protein, 12 aa, Artificial Sequence
  Synthetic modified hinge region(U3-HC9)
ERKCCVECPPCP

SEQ ID NO: 103 - protein, 14 aa, Artificial Sequence
  Synthetic modified hinge region(U6-HC8)
EPRDCGCKPCPPCP

SEQ ID NO: 104 - protein, 13 aa, Artificial Sequence
  Synthetic modified hinge region(U8-HC5)
EKCDKTHTCPPCP

SEQ ID NO: 105 - protein, 15 aa, Artificial Sequence
  Synthetic human hinge region
EPKSCDKTHTCPPCP

SEQ ID NO: 106 - protein, 17 aa, Artificial Sequence
  Synthetic CDR-L1 of antibody L3-11Y
KSSQSLLAWGNQNNYLA

SEQ ID NO: 107 - protein, 114 aa, Artificial Sequence
  Synthetic amino acid sequence of light chain variable region of antibody L3-11Y
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKR

SEQ ID NO: 108 - protein, 220 aa, Artificial Sequence
  Synthetic amino acid sequence of light chain of antibody L3-11Y
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 114 aa, Artificial Sequence
  Synthetic light chain variable region of anti c-Met antibody
DIQMTQSPSSLSASVGDRVTITCKSSQSLLASGNQNNYLAWYQQKPGKAPKMLIIWASTRVSGVPSRFSGSGSGTDFTLTISSLQPEDFATYYCQQSYSRPYTFGQGTKVEIKR

(19) United States
(12) Patent Application Publication
(10) Pub. No.: US 2017/0233489 A1
(43) Pub. Date: Aug. 17, 2017

(54) COMPOSITION FOR COMBINATION THERAPY COMPRISING ANTI-HER2 ANTIBODY AND ANTI-C-MET ANTIBODY

(71) Applicant: Samsung Electronics Co., Ltd., Suwon-si, Gyeonggi-do (KR)

(72) Inventors: Seon Hui SHIM, Yuseong-gu, Daejeon (KR); Jung Wook LEE, Yongin-si, Gyeonggi-do (KR)

(21) Appl. No.: 15/313,923

(22) PCT Filed: May 26, 2014
(86) PCT No.: PCT/KR2014/004672

§ 371 Date: Nov. 23, 2016

(51) Int. Cl. C07K 16/32; C07K 16/28

(52) U.S. Cl. CPC C07K 16/32; C07K 16/2863; A61K 2039/507

(57) ABSTRACT

A composition for combination therapy for preventing and/or treating a cancer, and a composition for combination therapy for inhibiting metastasis and/or angiogenesis, including an anti-c-Met antibody and an anti-HER2 antibody; and a method of preventing and/or treating a cancer and a method of inhibiting metastasis and/or angiogenesis, including co-administering an anti-c-Met antibody and an anti-HER2 antibody, are provided.

### BACKGROUND OF THE INVENTION

1. Field

A composition for combination therapy for preventing and/or treating a cancer, and a composition for combination therapy for inhibiting metastasis and/or angiogenesis, including an anti-c-Met antibody and an anti-HER2 antibody; and a method of preventing and/or treating a cancer and a method of inhibiting metastasis and/or angiogenesis, including co-administering an anti-c-Met antibody and an anti-HER2 antibody, are provided. 2. Description of the Related Art

c-Met, a typical receptor tyrosine kinase (RTK) existing on the surface of cells, binds to hepatocyte growth factor (HGF) to promote intracellular signal transduction and cell growth. c-Met overexpression is linked to cancer incidence, cancer metastasis, cancer cell migration, cancer cell penetration, angiogenesis, etc. In particular, cMet is known to be overexpressed in various solid cancers such as brain cancer, and the like, and directly involved in invasion and metastasis of cancers. Recently, it is being reported that c-Met overexpression causes resistance to the existing therapeutics. Thus, c-Met is becoming an important target in combination therapy.

HER2 (Human Epidermal growth factor Receptor 2 protein) is known to play an important role in controlling cell proliferation and differentiation. In particular, it has a strong tendency to be assembled into homo- and/or hetero-dimer with another HER receptor if extracellular growth factors bind thereto, which results in activations of various signal transduction routes, thus inducing apoptosis, survival or cell proliferation.

Among anti-HER2 antibodies, recombinant humanized versions referred to as huMAb4D5-8, rhuMAb HER2, Trastuzumab, or HERCEPTIN® (U.S. Pat. No. 5,821,337) are clinically active in ErbB2-overexpressing metastatic breast cancer patients who previously received extensive anticancer therapies (Baselga et al., J. Clin. Oncol. 14:737-744 (1996)). HERCEPTIN® was approved for sale for treatment of ErbB2 protein-overexpressing metastatic breast cancer patients at 1998. Despite treatment efficiency of Herceptin in breast cancers, the approval was strictly limited only for 30% of breast cancer patients where tumors overexpress HER2. The reason why 70% of breast cancer patients do not respond to Trastuzumab or insufficiently respond is that the individual tumor does not overexpress HER2, or exhibits resistance to Herceptin even though the tumor expresses HER2. Thus, there is a limitation in that an anti-HER2 mAb is inefficient in tumors where HER2 is low expressed or the overexpression disappears.

Strategies of the existing combination therapies are mainly combinations of compounds, or combinations of a compound and an antibody drug. Combinations with an antibody drug are being attempted to minimize side effects and inhibit only a specific molecule in cancer cells. However, combination therapy of two antibodies fails to achieve significant anticancer effects. Currently, representative examples of clinically progressed combinations of antibodies are Erbitux® and Avastin® in colorectal cancer, Herceptin® and Perjeta™ in breast cancer, and the like. Although the combination of Herceptin® and Perjeta™ in breast cancer led to significantly positive results, the combination of Erbitux® and Avastin® failed to achieve good anticancer effects as expected. And, since only a few antibody drugs are FDA approved, the number of combinations also is small. Even if combined, distinct combination effects may not be easily achieved.

Thus, there is a desire for an improved anticancer treatment.

### BRIEF SUMMARY OF THE INVENTION

Provided is a pharmaceutical composition for combination therapy, including (a) an anti-c-Met antibody or an antigen-binding fragment thereof and (b) an anti-HER2 antibody or an antigen-binding fragment thereof, wherein the anti-c-Met antibody or antigen-binding fragment thereof specifically binds to an epitope comprising 5 or more contiguous amino acids within the SEMA domain of c-Met protein. In an embodiment, the pharmaceutical composition for combination therapy may be used for preventing and/or treating a cancer and/or cancer metastasis. In another embodiment, the pharmaceutical composition for combination therapy may be used for inhibiting angiogenesis.

Provided is a method for prevention or treatment of a cancer or cancer metastasis, comprising co-administering (a) an anti-c-Met antibody or an antigen-binding fragment thereof and (b) an anti-HER2 antibody or an antigen-binding fragment thereof to a patient in need thereof, wherein the anti-c-Met antibody or the antigen-binding fragment thereof specifically binds to an epitope comprising 5 or more contiguous amino acids within the SEMA domain of c-Met protein.

Also provided is a method for inhibition of angiogenesis, comprising co-administering (a) an anti-c-Met antibody or an antigen-binding fragment thereof and (b) an anti-HER2 antibody or an antigen-binding fragment thereof to a patient in need thereof, wherein the anti-c-Met antibody or the antigen-binding fragment thereof specifically binds to an epitope comprising 5 or more contiguous amino acids within the SEMA domain of c-Met protein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 contains graphs demonstrating the expression of c-Met and HER2 in the SKBR3 (upper), MKN45 (middle), and NCI-N87 (lower) cancer cell lines.

FIG. 2 contains graphs providing the results of single treatment and combination treatment of anti-cMet antibody L3-1Y and anti-HER2 antibody Herceptin without HGF (upper) or with HGF (lower).

FIG. 3 contains photographs showing results confirming inhibition of migration according to single treatment and combination treatment of anti-cMet antibody L3-1Y and anti-HER2 antibody Herceptin.

FIG. 4 is a graph that demonstrates relative gene expression of genes after combination treatment of anti-cMet antibody and anti-HER2 antibody without (−) or with (+) HGF.

FIG. 5 is a graph that demonstrates the influence of combination treatment of anti-cMet antibody and anti-HER2 antibody on IL-8 protein level.

### DETAILED DESCRIPTION OF THE INVENTION

Applicants discovered that co-administration of an anti-c-Met antibody and an anti-Her 2 antibody achieves significant synergistic anticancer effects by simultaneously inhibiting growth and metastasis of cancer cells not only at low HGF expression state but also at high expression state, as compared to single administration of each antibody.

Thereby, Applicants discovered that the above explained limitation of the existing anti-HER2 antibodies (in that they are effective only for cancer and tumor tissues expressing low level or insignificant HGF) may be overcome. Furthermore, excellent effects may be achieved in terms of a decrease in pro-angiogenic factors, as well as synergistic anticancer effects through the co-administration, thus further increasing anticancer effects through inhibition of angiogenesis of cancer cells.

Accordingly, the present invention provides a combination therapy that may exert synergistic anticancer effects, which is particularly useful for patients with HGF overexpression who could not be treated by the existing anticancer agents, as well as patients with low HGF expression. Namely, the pharmaceutical composition according to one embodiment may achieve significantly improved anticancer effects through combination treatment with an anti-c-Met antibody in case acquired resistance occurs in cells sensitive to an anti-HER2 antibody by HGF.

Additionally, the combination therapy may decrease the required dose of anticancer drugs when compared to a single administration of each antibody, thus minimizing side effects and increasing convenience of patients.

Thus, one embodiment provides an anticancer composition for combination therapy comprising an anti-c-Met antibody and an anti-HER2 antibody as active ingredients.

The anticancer composition may preferably inhibit growth and/or metastasis of cancer cells. Specifically, if HGF exists, migration of cancer cells may increase, which may be inhibited by combination treatment of the two antibodies, thus achieving excellent effects of inhibiting metastasis of cancer.

The composition for combination therapy may decrease pro-angiogenic factors even at an HGF overexpression state equivalent to the level at HGF low expression state, thus achieving very excellent effect of inhibition of angiogenesis.

Genes whose expression may be changed by the combination treatment of an anti- c-Met antibody and an anti-Her 2 antibody were identified by Applicants (see Examples 4 and 5). Examples of the genes and proteins include IL-8. IL-8 is a potent pro-angiogenic factor together with VEGF, and may influence inhibition of angiogenesis in a tumor microenvironment.

In addition to IL-8, the pro-angiogenic factors may be at least one selected from the group consisting of uPAR, VEGF, Angiopoietin, IL-6 (Interleukin 6) and FGF, though preferably IL-8 (Interleukin 8), or uPAR (Urinary-type plasminogen activator receptor) exhibits a change in protein level similar to gene level.

Thus, the IL-8 or uPAR may be applied for a biomarker in serum, which may detect the effects of combination treatment of an anti-c-Met antibody and an anti-HER2 antibody.

Accordingly, another embodiment provides a pharmaceutical composition for inhibiting angiogenesis, containing an anti-c-Met antibody and an anti-HER2 antibody as active ingredients.

The anticancer composition for combination therapy and the pharmaceutical composition for inhibiting angiogenesis may be formulated as a combined mixture (e.g., a single composition comprising two or more active ingredients) by mixing an anti-c-Met antibody and an anti-HER2 antibody for co-administration. The anti-c-Met antibody and the anti-HER2 antibody can be present in any amount that is pharmaceutically effective when used together. The composition thus formulated can be used for simultaneous administration of the two active ingredients.

Alternatively, the anti-c-Met antibody and the anti-HER2 antibody may be respectively formulated in a separate composition, and the two active ingredients can be separately administered simultaneously or sequentially. For instance, a first pharmaceutical composition containing a pharmaceutically effective amount of an anti-c-Met antibody as an active ingredient, and a second pharmaceutical composition containing a pharmaceutically effective amount of an anti-HER2 antibody as an active ingredient can be administered simultaneously or sequentially. In the case of the sequential administration, any order of administration may be used.

Another embodiment provides a kit useful for prevention and/or treatment of cancers and/or cancer metastasis, including a first pharmaceutical composition containing an anti-c-Met antibody as an active ingredient, a second pharmaceutical composition containing an anti-HER2 antibody as an active ingredient, and a package container.

Still another embodiment provides a kit useful for inhibiting angiogenesis, including a first pharmaceutical composition containing an anti-c-Met antibody as an active ingredient, a second pharmaceutical composition containing an anti-HER2 antibody as an active ingredient, and a package container.

In the kit, the anti-c-Met antibody and the anti-HER2 antibody may be used in amounts that are pharmaceutically effective when combined, which amount may be determined by the skilled medical practitioner or medical researcher. The package container can be any container that holds or otherwise links the two compositions in individual containers together in a single unit (e.g., a box that holds both containers, or plastic wrap that binds both containers together), or the package container may be a single, divided container having at least two chambers that each hold one of the two compositions.

A method for prevention and/or treatment of cancers and/or cancer metastasis also is provided. The method includes co-administering an anti-c-Met antibody and an anti-HER2 antibody to a patient in need of prevention and/or treatment of cancers. The anti-c-Met antibody and the anti-HER2 antibody may be administered in amounts that are pharmaceutically effective when combined, which amount may be determined by the skilled medical practitioner or medical researcher. The method may further include, prior to the co-administration step, a step of identifying a patient in need of the prevention and/or treatment of cancer and/or cancer metastasis.

Still another embodiment provides a method for inhibition of angiogenesis, including co-administering a pharmaceutically effective amount of an anti-c-Met antibody and a pharmaceutically effective amount of an anti-HER2 antibody to a patient in need of inhibition of angiogenesis. The anti-c-Met antibody and the anti-HER2 antibody may be administered in amounts that are pharmaceutically effective when combined, which amount may be determined by the skilled medical practitioner or medical researcher. The method may further include, prior to the co-administration step, a step of identifying a patient in need of the inhibition of angiogenesis.

In the method of prevention and/or treatment of cancer and/or cancer metastasis, and/or method for inhibition of angiogenesis, the co-administration may be conducted by administering a combined mixture of a pharmaceutically effective amount of an anti-c-Met antibody and a pharmaceutically effective amount of an anti-HER2 antibody. According to another embodiment, the co-administration may include simultaneously or sequentially conducting a first step of administering a pharmaceutically effective amount of an anti-c-Met antibody as an active ingredient, and a second step of administering a pharmaceutically effective amount of an anti-HER2 antibody as an active ingredient. In the case of the sequential administration, the first step and the second step may be performed in any order.

The patients may be mammals including primates, such as humans and monkeys, and rodents, such as mice and rats. Furthermore, the patients may be cancer patients, or patients having a high HGF level. In a specific embodiment, if cancer tissues exist, since HGF is secreted by stromal cells existing in the cancer tissues, total HGF level may be influenced by HGF secreted by the surrounding cells, as well as HGF secreted by cancer cells themselves, and thus increased. The increased HGF level may lower the effects of drugs such as herceptin, but the anticancer composition for combination therapy according to one embodiment may overcome the problem of acquired resistance due to HGF, thus achieving significant synergistic effects.

Accordingly, the method for prevention and/or treatment of cancers or the method for inhibition of angiogenesis may further include identifying a patient in need of prevention and/or treatment of cancers, or inhibition of angiogenesis, before the administration step.

The step of identifying the patient may include, before the administration step, (1) comparing the HGF expression level of a cell sample separated from the patient with the HGF expression level of normal persons (i.e., a negative control); and (2) if the HGF expression level of a cell sample separated from the patient is higher than the HGF expression level of normal persons (i.e., a negative control), deciding the patient from which the cell sample originated is a candidate for administration of the combination therapy (e.g., for the treatment of cancers or the inhibition of angiogenesis).

The identification of the step (1) may be conducted by methods well-known in the art and, for example, serum may be separated from blood, and the HGF expression amount therein may be confirmed by ELISA, and the like.

The composition for combination therapy and/or the method of combination therapy according to the present invention may be used for prevention and/or treatment of cancers. The effects of prevention and/or treatment of cancers may include inhibition of aggravation of cancers due to migration, invasion, metastasis, and the like, and angiogenesis of cancer cells, as well as inhibition of growth of cancer cells.

The cancer may be related to overexpression and/or abnormal activation of c-Met and/or HER2. The cancer may include solid cancers and blood cancers. The cancer may be, although not limited thereto, at least one selected from the group consisting of squamous cell carcinoma, small-cell lung cancer, non-small-cell lung cancer, adenocarcinoma of the lung, squamous cell carcinoma of the lung, peritoneal carcinoma, skin cancer, melanoma in the skin or eyeball, rectal cancer, cancer near the anus, esophagus cancer, small intestinal tumor, endocrine gland cancer, parathyroid cancer, adrenal cancer, soft-tissue sarcoma, urethral cancer, chronic or acute leukemia, lymphocytic lymphoma, hepatoma, gastric cancer, gastrointestinal cancer, pancreatic cancer, glioblastoma, cervical cancer, ovarian cancer, liver cancer, bladder cancer, hepatocellular adenoma, breast cancer, colon cancer, large intestine cancer, endometrial carcinoma or uterine carcinoma, salivary gland tumor, kidney cancer, prostate cancer, vulvar cancer, thyroid cancer, head and neck cancers, osteosarcoma, and the like, preferably, gastric cancer, lung cancer or breast cancer. The cancer may include metastatic cancers, as well as primary cancers.

And, the composition for combination therapy and/or the method of combination therapy may be for inhibition of angiogenesis of cancer cells as explained above.

The “pharmaceutically effective amount” refers to an administration amount required to achieve aimed effects, for example, anticancer effects including inhibition of growth and metastasis of cancer cells, or decrease in angiogenesis factors and the resulting inhibition of angiogenesis, and it may be appropriately prescribed depending on aimed effects, types and severity of diseases or symptoms, patient conditions, administration route, dosage form, and the like.

Unless stated otherwise, the anti c-Met antibody included in the anticancer composition for combination therapy may refer to not only a whole type anti-c-Met antibody but also antigen-binding fragments or variants of the antibody. The antigen-binding fragment of the anti-c-Met antibody may refer to a fragment including an antigen-binding region of the anti-c-Met antibody, and can be selected from the group consisting of a complementarity determining region (CDR), fragment including CDR and Fc region, scFv, (scFv)₂, Fab, Fab', and F(ab')₂ of the anti-c-Met antibody. The variant of the antibody may be any isotype of antibodies derived from human (e.g., IgA, IgD, IgE, IgG (IgG1, IgG2, IgG3, IgG4), IgM, etc.) and other animals found in nature and/or one including any Fc region of antibodies derived from human and other animals, having a mutated hinge, wherein at least one (e.g., two, three, four, five, six, seven, eight, nine, or ten) amino acid is changed, deleted, inserted, or added.

The anti-c-Met antibody may recognize a specific region of c-Met, e.g., a specific region in the SEMA domain, as an epitope. It may be any antibody or antigen-binding fragment that acts on c-Met to induce c-Met intracellular internalization and degradation.

c-Met, a receptor for hepatocyte growth factor (HGF), may be divided into three portions: extracellular, transmembrane, and intracellular. The extracellular portion is composed of an a-subunit and a (3-subunit which are linked to each other through a disulfide bond, and contains a SEMA domain responsible for binding HGF, a PSI domain (plexin-semaphorins-integrin homology domain) and an IPT domain (immunoglobulin-like fold shared by plexins and transcriptional factors domain). The SEMA domain of c-Met protein may include the amino acid sequence of SEQ ID NO: 79, and is an extracellular domain that functions to bind HGF. A specific region of the SEMA domain, that is, a region including the amino acid sequence of SEQ ID NO: 71, which corresponds to a range from amino acid residues 106 to 124 of the amino acid sequence of the SEMA domain (SEQ ID NO: 79) of c-Met protein, is a loop region between the second and the third propellers within the epitopes of the SEMA domain. The region acts as an epitope for the specific anti-c-Met antibody of the present invention.

The term “epitope” as used herein, refers to an antigenic determinant, a part of an antigen recognized by an antibody. In one embodiment, the epitope may be a region including 5 or more contiguous amino acid residues within the SEMA domain (SEQ ID NO: 79) of c-Met protein, for instance, 5 to 19 contiguous amino acid residues within the amino acid sequence of SEQ ID NO: 71. For example, the epitope may be a polypeptide including 5 to 19 contiguous amino acids selected from among partial combinations of the amino acid sequence of SEQ ID NO: 71, wherein the polypeptide essentially includes the amino sequence of SEQ ID NO: 73 (EEPSQ) serving as an essential element for the epitope. For example, the epitope may be a polypeptide comprising, consisting essentially of, or consisting of the amino acid sequence of SEQ ID NO: 71, SEQ ID NO: 72, or SEQ ID NO: 73. As used herein, the term “contiguous amino acid residues” may refer to contiguous amino acid residues on the primary, secondary, or tertiary structure of a protein.

The epitope including the amino acid sequence of SEQ ID NO: 72 corresponds to the outermost part of the loop between the second and third propellers within the SEMA domain of a c-Met protein. The epitope including the amino acid sequence of SEQ ID NO: 73 is a site to which the antibody or antigen-binding fragment according to one embodiment most specifically binds.

Thus, the anti-c-Met antibody may specifically bind to an epitope which includes 5 to 19 contiguous amino acids selected from among partial combinations of the amino acid sequence of SEQ ID NO: 71, including SEQ ID NO: 73 as an essential element. For example, the anti-c-Met antibody may specifically bind to an epitope comprising the amino acid sequence of SEQ ID NO: 71, SEQ ID NO: 72, or SEQ ID NO: 73.

In one embodiment, the anti-c-Met antibody may be an antibody or an antigen-binding fragment thereof, which comprises:

(a) at least one heavy chain complementarity determining region (CDR) selected from the group consisting of (i) a CDR-H1 comprising the amino acid sequence of SEQ ID NO: 4; (ii) a CDR-H2 comprising the amino acid sequence of SEQ ID NO: 5, the amino acid sequence of SEQ ID NO: 2, or an amino acid sequence comprising 8-19 consecutive amino acids of the amino acid sequence of SEQ ID NO: 2, wherein the 8-19 consecutive amino acids comprise amino acid residues from the 3rd to 10th positions of the amino acid sequence SEQ ID NO: 2; and (iii) a CDR-H3 comprising the amino acid sequence of SEQ ID NO: 6, the amino acid sequence of SEQ ID NO: 85, or an amino acid sequence comprising 6-13 consecutive amino acids of the amino acid sequence of SEQ ID NO: 85, wherein the 6-13 consecutive amino acids comprise amino acid residues from the 1st to 6th positions of the amino acid sequence of SEQ ID NO: 85, or a heavy chain variable region comprising the at least one heavy chain CDR;

(b) a light chain variable region comprising at least one light chain CDR selected from the group consisting of (i) a CDR-L1 comprising the amino acid sequence of SEQ ID NO: 7, (b) a CDR-L2 comprising the amino acid sequence of SEQ ID NO: 8, and (c) a CDR-L3 comprising the amino acid sequence of SEQ ID NO: 9, the amino acid sequence of SEQ ID NO: 86, or an amino acid sequence comprising 9-17 consecutive amino acids of the amino acid sequence of SEQ ID NO: 89, wherein the 9-17 consecutive amino acids comprise amino acid residues from the 1st to 9th positions of the amino acid sequence of SEQ ID NO: 89, or a light chain variable region comprising the at least one light chain CDR;

(c) a combination of the at least one heavy chain CDR and the at least one light chain CDR; or

(d) a combination of the heavy chain variable region and the light chain variable region.

Herein, the amino acid sequences of SEQ ID NOS: 4 to 9 are respectively represented by following Formulas I to VI, below:

Formula I: Xaa₁-Xaa₂-Tyr-Tyr-Met-Ser (SEQ ID NO: 4), wherein Xaa₁ is absent or Pro or Ser, and Xaa₂ is Glu or Asp,

Formula II: Arg-Asn-Xaa₃-Xaa₄-Asn-Gly-Xaa₈-Thr (SEQ ID NO: 5), wherein Xaa₃ is Asn or Lys, Xaa4 is Ala or Val, and Xaa₅ is Asn or Thr,

Formula III: Asp-Asn-Trp-Leu-Xaa₆-Tyr (SEQ ID NO: 6), wherein Xaa₆ is Ser or Thr,

Formula IV: Lys-Ser-Ser-Xaa₇-Ser-Leu-Leu-Ala-Xaa₈-Gly-Asn-Xaa₉-Xaa₁₀-Asn-Tyr-Leu-Ala (SEQ ID NO: 7), wherein Xaa ₇ is His, Arg, Gln, or Lys, Xaa₈ is Ser or Trp, Xaa₉ is His or Gln, and Xaa₁₀ is Lys or Asn,

Formula V: Trp-Xaa₁₁-Ser-Xaa₁₂-Arg-Val-Xaa₁₃ (SEQ ID NO: 8), wherein Xaa₁₁ is Ala or Gly, Xaa₁₂ is Thr or Lys, and Xaa₁₃ is Ser or Pro, and

Formula VI: Xaa₁₄-Gln-Ser-Tyr-Ser-Xaa₁₅-Pro-Xaa₁₆-Thr (SEQ ID NO: 9), wherein Xaa₁₄ is Gly, Ala, or Gln, Xaa₁₅ is Arg, His, Ser, Ala, Gly, or Lys, and Xaa₁₆ is Leu, Tyr, Phe, or Met.

In one embodiment, the CDR-H1 may comprise an amino acid sequence selected from the group consisting of SEQ ID NOS: 1, 22, 23, and 24. The CDR-H2 may include an amino acid sequence selected from the group consisting of SEQ ID NOS: 2, 25, and 26. The CDR-H3 may include an amino acid sequence selected from the group consisting of SEQ ID NOS: 3, 27, 28, and 85.

The CDR-L1 may include an amino acid sequence selected from the group consisting of SEQ ID NOS: 10, 29, 30, 31, 32, 33, and 106. The CDR-L2 may include an amino acid sequence selected from the group consisting of SEQ ID NOS: 11, 34, 35, and 36. The CDR-L3 may include an amino acid sequence selected from the group consisting of SEQ ID NOS: 12, 13, 14, 15, 16, 37, 86, and 89.

In another embodiment, the antibody or the antigen-binding fragment may comprise (a) a heavy variable region comprising (i) a polypeptide (CDR-H1) comprising an amino acid sequence selected from the group consisting of SEQ ID NOS: 1, 22, 23, and 24, (ii) a polypeptide (CDR-H2) comprising an amino acid sequence selected from the group consisting of SEQ ID NOS: 2, 25, and 26, and (iii) a polypeptide (CDR-H3) comprising an amino acid sequence selected from the group consisting of SEQ ID NOS: 3, 27, 28, and 85; and (b) a light variable region comprising (i) a polypeptide (CDR-L1) comprising an amino acid sequence selected from the group consisting of SEQ ID NOS: 10, 29, 30, 31, 32, 33 and 106, (ii) a polypeptide (CDR-L2) comprising an amino acid sequence selected from the group consisting of SEQ ID NOS: 11, 34, 35, and 36, and (iii) a polypeptide (CDR-L3) comprising an amino acid sequence selected from the group consisting of SEQ ID NOS 12, 13, 14, 15, 16, 37, 86, and 89.

The term “c-Met” or “c-Met protein” refers to a receptor tyrosine kinase (RTK) which binds hepatocyte growth factor (HGF). c-Met may be a c-Met protein from any species, particularly a mammal, for instance, primates such as human c-Met (e.g., GenBank Accession Number NP_000236) or monkey c-Met (e.g., Macaca mulatta, GenBank Accession Number NP_001162100), or rodents such as mouse c-Met (e.g., GenBank Accession Number NP_032617.2) or rat c-Met (e.g., GenBank Accession Number NP_113705.1), and the like. The c-Met protein may include a polypeptide encoded by the nucleotide sequence identified as GenBank Accession Number NM_000245, a polypeptide including the amino acid sequence identified as GenBank Accession Number NP_000236 or extracellular domains thereof. The receptor tyrosine kinase c-Met participates in various mechanisms, such as cancer development, metastasis, migration of cancer cells, invasion of cancer cells, angiogenesis, and the like.

Animal-derived antibodies produced by immunizing non-immune animals with a desired antigen generally invoke immunogenicity when injected into humans for the purpose of medical treatment and, thus, chimeric antibodies have been developed to inhibit such immunogenicity. Chimeric antibodies are prepared by replacing constant regions of animal-derived antibodies that cause an anti-isotype response with constant regions of human antibodies by genetic engineering. Chimeric antibodies are considerably improved in an anti-isotype response compared to animal-derived antibodies, but animal-derived amino acids still have variable regions, so that chimeric antibodies have side effects with respect to a potential anti-idiotype response. Humanized antibodies have been developed to reduce such side effects. Humanized antibodies are produced by grafting complementarity determining regions (CDRs), which serve an important role in antigen binding in variable regions of chimeric antibodies, into a human antibody framework.

The most important thing in CDR grafting to produce humanized antibodies is choosing the optimized human antibodies for accepting CDRs of animal-derived antibodies. Antibody databases, analysis of a crystal structures, and technology for molecule modeling are used. However, even when the CDRs of animal-derived antibodies are grafted to the most optimized human antibody framework, amino acids positioned in a framework of the animal-derived CDRs affecting antigen binding are present. Therefore, in many cases, antigen binding affinity is not maintained and, thus, application of additional antibody engineering technology for recovering the antigen binding affinity is necessary.

The anti c-Met antibodies may be mouse-derived antibodies, mouse-human chimeric antibodies, humanized antibodies, or human antibodies. The antibodies or antigen-binding fragments thereof may be isolated from a living body or non-naturally occurring. The antibodies or antigen-binding fragments thereof may be synthetic or recombinant.

An intact antibody includes two full-length light chains and two full-length heavy chains, in which each light chain is linked to a heavy chain by disulfide bonds. The antibody includes a heavy chain constant region and a light chain constant region. The heavy chain constant region is of a gamma (γ), mu (μ), alpha (α), delta (δ), or epsilon (ε) type, which may be further categorized as gamma 1 (γ1), gamma 2 (γ2), gamma 3 (γ3), gamma 4 (γ4), alpha 1 (α1), or alpha 2 (α2). The light chain constant region is of either a kappa (κ) or lambda (λ) type.

As used herein, the term “heavy chain” refers to full-length heavy chain, and fragments thereof, including a variable region VH that includes amino acid sequences sufficient to provide specificity to antigens, and three constant regions, CH1, CH2, and CH3, and a hinge. The term “light chain” refers to a full-length light chain and fragments thereof, including a variable region VL that includes amino acid sequences sufficient to provide specificity to antigens, and a constant region CL.

The term “complementarity determining region (CDR)” refers to an amino acid sequence found in a hyper variable region of a heavy chain or a light chain of immunoglobulin. The heavy and light chains may respectively include three CDRs (CDR-H1, CDR-H2, and CDR-H3; and CDR-L1, CDR-L2, and CDR-L3). The CDRs may provide contact residues that play an important role in the binding of antibodies to antigens or epitopes. The terms “specifically binding” and “specifically recognized” are well known to one of ordinary skill in the art, and indicate that an antibody and an antigen specifically interact with each other to lead to an immunological activity.

The antibody used herein includes an antigen-binding fragment as well as an intact antibody. The term “antigen-binding fragment” used herein refers to fragments of an intact immunoglobulin including portions of a polypeptide comprising antigen-binding regions having the ability to specifically bind to the antigen. In one embodiment, the antibody may be an antigen-binding fragment selected from the group consisting of scFv, (scFv)₂, Fab, Fab′, and F(ab′)₂.

Among the antigen-binding fragments, Fab that includes light chain and heavy chain variable regions, a light chain constant region, and a first heavy chain constant region CHI, includes one antigen-binding site.

The Fab′ fragment is different from the Fab fragment in that Fab′ includes a hinge region with at least one cysteine residue at the C-terminal of CH1.

The F(ab′)₂ antibody is formed through disulfide bridging of the cysteine residues in the hinge region of the Fab′ fragment. Fv is the smallest antibody fragment with only a heavy chain variable region and a light chain variable region. Recombination techniques of generating the Fv fragment are widely known in the art.

Two-chain Fv includes a heavy chain variable region and a light chain region which are linked by a non-covalent bond. Single-chain Fv generally includes a heavy chain variable region and a light chain variable region which are linked by a covalent bond via a peptide linker or linked at the C-terminals to have a dimer structure like the two-chain Fv.

The antigen-binding fragments may be attainable using protease (for example, the Fab fragment may be obtained by restricted cleavage of a whole antibody with papain, and the F(ab′)₂ fragment may be obtained by cleavage with pepsin), or may be prepared by using a genetic recombination technique.

The term “hinge region,” as used herein, refers to a region between CH1 and CH2 domains within the heavy chain of an antibody which functions to provide flexibility for the antigen-binding site.

When an animal antibody undergoes a chimerization process, the IgG1 hinge of animal origin may be replaced with a human IgG1 hinge or IgG2 hinge while the disulfide bridges between two heavy chains are reduced from three to two in number. In addition, an animal-derived IgG1 hinge is shorter than a human IgG1 hinge. Accordingly, the rigidity of the hinge is changed. Thus, a modification of the hinge region may bring about an improvement in the antigen binding efficiency of the humanized antibody. The modification of the hinge region through amino acid deletion, addition, or substitution is well-known to those skilled in the art.

In one embodiment, the anti-c-Met antibody or an antigen-binding fragment thereof may be modified by the deletion, insertion, addition, or substitution of at least one (e.g., two, three, four, five, six, seven, eight, nine, or ten) amino acid residue in the amino acid sequence of the hinge region so that it exhibits enhanced antigen-binding efficiency. For example, the antibody may include a hinge region comprising the amino acid sequence of SEQ ID NO: 100 (U7-HC6), 101 (U6-HC7), 102 (U3-HC9), 103 (U6-HC8), or 104 (U8-HC5), or a hinge region comprising the amino acid sequence of SEQ ID NO: 105 (non-modified human hinge). Preferably, the hinge region includes the amino acid sequence of SEQ ID NO: 100 or 101.

In one embodiment of the anti-c-Met antibody or antigen-binding fragment, the variable domain of the heavy chain comprises the amino acid sequence of SEQ ID NO: 17, 74, 87, 90, 91, 92, 93, or 94 and the variable domain of the light chain comprises the amino acid sequence of SEQ ID NO: 18, 19, 20, 21, 75, 88, 95, 96, 97, 98, 99, or 107.

In one embodiment, the anti-c-Met antibody may be a monoclonal antibody. The monoclonal antibody may be produced by the hybridoma cell line deposited with the Korean Cell Line Research Foundation under Accession No. KCLRF-BP-00220, which binds specifically to the extracellular region of c-Met protein (refer to Korean Patent Publication No. 2011-0047698). The anti-c-Met antibody may include all the antibodies defined in Korean Patent Publication No. 2011-0047698.

By way of further example, the anti-c-Met antibody or the antibody fragment may include:

(a) a heavy chain comprising an amino acid sequence selected from the group consisting of (i) the amino acid sequence of the amino acid sequence of SEQ ID NO: 62 (wherein amino acid residues from the 1st to 17th positions of the amino acid sequence of SEQ ID NO: 62 correspond to a signal peptide), (ii) the amino acid sequence from the 18th to 462nd positions of the amino acid sequence of SEQ ID NO: 62, (iii) the amino acid sequence of SEQ ID NO: 64 (wherein amino acid residues from the 1st to 17th positions of the amino acid sequence of SEQ ID NO: 64 correspond to a signal peptide), (iv) the amino acid sequence from the 18th to 461st positions of the amino acid sequence of SEQ ID NO: 64, (v) the amino acid sequence of SEQ ID NO: 66 (wherein amino acid residues from the 1st to 17th positions of the amino acid sequence of SEQ ID NO: 66 correspond to a signal peptide), and (vi) the amino acid sequence from the 18th to 460th positions of SEQ ID NO: 66; and

(b) a light chain comprising an amino acid sequence selected from the group consisting of (i) the amino acid sequence of SEQ ID NO: 68 (wherein amino acid residues from the 1st to 20th positions of the amino acid sequence of SEQ ID NO: 68 correspond to a signal peptide), (ii) the amino acid sequence from the 21st to 240th positions of the amino acid sequence of SEQ ID NO: 68, (iii) the amino acid sequence of SEQ ID NO: 70 (wherein amino acid residues from the 1st to 20th positions of the amino acid sequence of SEQ ID NO: 70 correspond to a signal peptide), (iv) the amino acid sequence from the 21st to 240th positions of the amino acid sequence of SEQ ID NO: 70, and (v) the amino acid sequence of SEQ ID NO: 108.

For example, the anti-c-Met antibody may be selected from the group consisting of:

an antibody comprising (a) a heavy chain comprising (i) the amino acid sequence of SEQ ID NO: 62 or (ii) the amino acid sequence from the 18th to 462nd positions of the amino acid sequence of SEQ ID NO: 62 and (b) a light chain comprising (i) the amino acid sequence of SEQ ID NO: 68 or (ii) the amino acid sequence from the 21st to 240th positions of the amino acid sequence of SEQ ID NO: 68;

an antibody comprising (a) a heavy chain comprising (i) the amino acid sequence of SEQ ID NO: 64 or (ii) the amino acid sequence from the 18th to 461st positions of the amino acid sequence of SEQ ID NO: 64 and (b) a light chain comprising (i) the amino acid sequence of the amino acid sequence of SEQ ID NO: 68 or (ii) the amino acid sequence from the 21st to 240th positions of the amino acid sequence of SEQ ID NO: 68;

an antibody comprising (a) a heavy chain comprising (i) the amino acid sequence of SEQ ID NO: 66 or (ii) the amino acid sequence from the 18th to 460th positions of the amino acid sequence of SEQ ID NO: 66 and (b) a light chain comprising (i) the amino acid sequence of SEQ ID NO: 68 or (ii) the amino acid sequence from the 21st to 240th positions of the amino acid sequence of SEQ ID NO: 68;

an antibody comprising (a) a heavy chain comprising (i) the amino acid sequence of SEQ ID NO: 62 or the amino acid sequence from the 18th to 462nd positions of the amino acid sequence of SEQ ID NO: 62 and (b) a light chain comprising (i) the amino acid sequence of SEQ ID NO: 70 or (ii) the amino acid sequence from the 21st to 240th positions of the amino acid sequence of SEQ ID NO: 70;

an antibody comprising (a) a heavy chain comprising (i) the amino acid sequence of SEQ ID NO: 64 or (ii) the amino acid sequence from the 18th to 461st positions of the amino acid sequence of SEQ ID NO: 64 and (b) a light chain comprising (i) the amino acid sequence of SEQ ID NO: 70 or (ii) the amino acid sequence from the 21st to 240th positions of the amino acid sequence of SEQ ID NO: 70;

an antibody comprising (a) a heavy chain comprising (i) the amino acid sequence of SEQ ID NO: 66 or (ii) the amino acid sequence from the 18th to 460th positions of the amino acid sequence of SEQ ID NO: 66 and (b) a light chain comprising (i) the amino acid sequence of SEQ ID NO: 70 or the amino acid sequence from the 21st to 240th positions of the amino acid sequence of SEQ ID NO: 70;

an antibody comprising (a) a heavy chain comprising (i) the amino acid sequence of SEQ ID NO: 62 or (ii) the amino acid sequence from the 18th to 462nd positions of the amino acid sequence of SEQ ID NO: 62 and (b) a light chain comprising the amino acid sequence of SEQ ID NO: 108;

an antibody comprising (a) a heavy chain comprising (i) the amino acid sequence of SEQ ID NO: 64 or (ii) the amino acid sequence from the 18th to 461st positions of the amino acid sequence of SEQ ID NO: 64 and (b) a light chain comprising the amino acid sequence of SEQ ID NO: 108; and

an antibody comprising (a) a heavy chain comprising (i) the amino acid sequence of SEQ ID NO: 66 or (ii) the amino acid sequence from the 18th to 460th positions of the amino acid sequence of SEQ ID NO: 66 and (b) a light chain comprising (i) the amino acid sequence of SEQ ID NO: 108.

The polypeptide of SEQ ID NO: 70 is a light chain comprising the human kappa (κ) constant region, and the polypeptide comprising the amino acid sequence of SEQ ID NO: 68 is a polypeptide obtained by replacing histidine at position 62 (corresponding to position 36 of the amino acid sequence of SEQ ID NO: 68 according to kabat numbering) of the polypeptide with the amino acid sequence of SEQ ID NO: 70 with tyrosine. The production yield of the antibodies may be increased by the replacement. The polypeptide with the amino acid sequence of SEQ ID NO: 108 is a polypeptide obtained by replacing serine at position 32 (position 27e according to kabat numbering in the amino acid sequence from amino acid residues 21 to 240 of the amino acid sequence of SEQ ID NO: 68; positioned within CDR-L1) of the amino acid sequence of SEQ ID NO: 108 with tryptophan. By such replacement, antibodies and antibody fragments including such sequences exhibit increased activities, such as c-Met biding affinity, c-Met degradation activity, Akt phosphorylation inhibition, and the like.

In another embodiment, the anti c-Met antibody may comprise a light chain complementarity determining region including the amino acid sequence of SEQ ID NO: 106, a light chain variable region including the amino acid sequence of SEQ ID NO: 107, or a light chain including the amino acid sequence of SEQ ID NO: 108.

According to one particular embodiment, the antibody may act as an antagonist of c-Met protein.

As used herein, the term “antagonist” is intended to encompass all molecules that at least partially block, suppress, or neutralize at least one of the biological activities of a target (e.g., c-Met). By way of example, an “antagonist” antibody means an antibody that represents suppression or inhibition of the biological activity of the antigen to which the antibody binds (e.g., c-Met). An antagonist may function to reduce ligand-induced receptor phosphorylation or to incapacitate or kill cells which have been activated by ligands. Also, an antagonist may completely interfere with receptor-ligand interaction or substantially reduce the interaction by changing the three-dimensional structure of the receptor or by down regulation.

The anti-HER2 antibody that is used for the composition of combination therapy for anticancer treatment and angiogenesis inhibition, unless otherwise described, means an antibody or an antigen binding fragment.

The anti-HER2 antibody may be selected from the group consisting of Trastuzumab, Pertuzumab, Trastuzumab emtansine (T-DM1), and a combination thereof, preferably Trastuzumab (product name: Herceptin).

The anticancer composition and the composition for inhibiting angiogenesis comprising an anti-c-Met antibody and an anti-HER2 antibody may be formulated as a unit dosage form using a pharmaceutically acceptable carrier and/or excipient, or it may be formulated to be contained to a multiple dosage container, according to a method that can be easily practiced by one of ordinary knowledge in the art. They may be formulated into a dosage form of a solution in oil or an aqueous medium, a suspension, a syrup, an emulsion, an extract, powders, granules, a tablet or a capsule, and may further include a dispersing agent or a stabilizing agent.

And, the anticancer composition and the composition for inhibiting angiogenesis including an anti-c-Met antibody and an anti-HER2 antibody may be separately administered as a single medicine, or co-administered with other medicines, and may be sequentially or simultaneously administered with conventional medicines.

Meanwhile, the composition may be formulated into an immunoliposome since it contains an antibody or an antigen binding fragment. A liposome containing an antibody may be prepared using any methods well-known in the art. The immunnoliposome may be a lipid composition including phosphatidylcholine, cholesterol, and polyethyleneglycol-derived phosphatidylethanolamine, and may be prepared by a reverse phase evaporation method. For example, Fab′ fragments of an antibody may be conjugated to the liposome through a disulfide-exchange reaction. A chemical drug, such as doxorubicin, may be further included in the liposome.

The combined mixture obtained by mixing a pharmaceutically effective amount of an anti-c-Met antibody and a pharmaceutically effective amount of an anti-HER2 antibody, a first pharmaceutical composition containing a pharmaceutically effective amount of an anti-c-Met antibody as an active ingredient, or a second pharmaceutical composition containing a pharmaceutically effective amount of an anti-HER2 antibody as an active ingredient may be provided together with a pharmaceutically acceptable carrier, diluent, and/or excipient.

The pharmaceutically acceptable carriers that may be included in the combined mixture or the pharmaceutical compositions may be those commonly used in formulations of drugs, and may be, but not limited to, at least one selected from the group consisting of lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and mineral oil. Besides these components, the combined mixture or the pharmaceutical compositions may further include at least one selected from the group consisting of a diluent, an excipient, a lubricant, a wetting agent, a sweetener, a flavor enhancer, an emulsifying agent, a suspension agent, and a preservative.

The mixture or the pharmaceutical compositions may be administered orally or parenterally. Parenteral administration may include intravenous injection, subcutaneous injection, muscular injection, intraperitoneal injection, endothelial administration, local administration, intranasal administration, intrapulmonary administration, and rectal administration. Since oral administration leads to digestion of proteins or peptides, an active ingredient in the compositions for oral administration must be coated or formulated to prevent digestion in stomach. In addition, the compositions may be administered using an optional device that enables an active substance to be delivered to target cells.

The term “the pharmaceutically effective amount” as used in this specification refers to an amount at which each active ingredient can exert pharmaceutically significant effects.

For single dose, a pharmaceutically effective amount of the anti-c-Met antibody and a pharmaceutically effective amount of the anti-HER2 antibody may be prescribed in various ways, depending on many factors including formulation methods, administration manners, ages, body weight, gender, pathologic conditions, diets of patients, administration time, administration interval, administration route, excretion speed, and reaction sensitivity. For example, the effective amount of the anti-c-Met antibody for single dose may be, but not limited to, in the range of 0.01 to 100 mg/kg, specifically 0.2 to 10 mg/kg, and the effective amount of the anti-HER2 antibody for single dose may be in the range of 0.01 to 100 mg/kg, particularly 0.2 to 10 mg/kg. The effective amount for a single dose may be formulated into a single formulation in a unit dosage form or formulated in suitably divided dosage forms, or it may be manufactured to be contained in a multiple dosage container. For the kit, the effective amount of the anti-c-Met antibody and the effective amount of the anti-HER2 antibody for single dose may be contained in a package container as a base unit.

The co-administration interval between the administrations that is defined as a period between the first administration and the following administration may be, but not limited to, 24 hours to 30 days, specifically 7 days to 14 days. The first pharmaceutical composition containing a pharmaceutically effective amount of an anti-c-Met antibody and the second pharmaceutical composition containing a pharmaceutically effective amount of an anti-HER2 antibody may be co-administered at a predetermined time interval (for example, several minutes, several hours, or several days, or several weeks) depending on types of diseases, patient conditions, and the like. For example, the first pharmaceutical composition and the second pharmaceutical composition may be simultaneously (administration interval within 1 minute) or sequentially (administration interval of 1 minute or more) administered. If they are sequentially administered, the administration interval between the first pharmaceutical composition and the second pharmaceutical composition may be 1 minute to 30 days, specifically 1 minute to 7 days, a minute to 24 hours, or 1 minute to 60 minutes, more specifically 1 to 10 minutes, and the administration order may be reversed.

The combined mixture or the pharmaceutical compositions may be a solution in oil or an aqueous medium, a suspension, a syrup, an emulsifying solution form, or they may be formulated into a form of an extract, elixirs, powders, granules, a tablet or a capsule. The combined mixture or the pharmaceutical compositions may further include a dispersing agent or a stabilizing agent for their formulation.

In particular, the anti-c-Met antibody and the anti-HER2 antibody, or the pharmaceutical composition containing a pharmaceutically effective amount thereof, may be formulated into an immunoliposome as described above.

The combination therapy of an anti-c-Met antibody and an anti-HER2 antibody according to the present invention enables effective anticancer treatment even for cancer cells where HGF is overexpressed, and thereby, excellent synergistic anticancer effects may be expected in cancer cells where anticancer effects may not be achieved or may be insignificant by single administration.

The anticancer effects may include effects of not only suppressing the growth of the cancer cells but also suppressing metastasis thereof, and furthermore, the combination therapy of the anti-c-Met antibody and the anti-HER2 antibody may effectively inhibit angiogenesis, thus further increasing anticancer activity.

Hereafter, the present invention will be described in detail by examples.

The following examples are intended merely to illustrate the invention and are not construed to restrict the invention.

### EXAMPLES

### Reference Example 1: Construction of Anti-c-Met Antibody

### 1.1. Production of “AbF46”, a Mouse Antibody to c-Met

### 1.1.1. Immunization of Mouse

To obtain immunized mice necessary for the development of a hybridoma cell line, each of five BALB/c mice (Japan SLC, Inc.), 4 to 6 weeks old, was intraperitoneally injected with a mixture of 100 μg of human c-Met/Fc fusion protein (R&D Systems) and one volume of complete Freund's adjuvant. Two weeks after the injection, a second intraperitoneal injection was conducted on the same mice with a mixture of 50 μg of human c-Met/Fc protein and one volume of incomplete Freund's adjuvant. One week after the second immunization, the immune response was finally boosted. Three days later, blood was taken from the tails of the mice and the sera were 1/1000 diluted in PBS and used to examine a titer of antibody to c-Met by ELISA. Mice found to have a sufficient antibody titer were selected for use in the cell fusion process.

### 1.1.2. Cell Fusion and Production of Hybridoma

Three days before cell fusion, BALB/c mice (Japan SLC, Inc.) were immunized with an intraperitoneal injection of a mixture of 50 m of human c-Met/Fc fusion protein and one volume of PBS. The immunized mice were anesthetized before excising the spleen from the left half of the body. The spleen was meshed to separate splenocytes which were then suspended in a culture medium (DMEM, GIBCO, Invitrogen). The cell suspension was centrifuged to recover the cell layer. The splenocytes thus obtained (1×10⁸ cells) were mixed with myeloma cells (Sp2/0) (1×10⁸ cells), followed by spinning to give a cell pellet. The cell pellet was slowly suspended, treated with 45% polyethylene glycol (PEG) (1 mL) in DMEM for 1 min at 37° C., and supplemented with 1 mL of DMEM. To the cells was added 10 mL of DMEM over 10 min, after which incubation was conducted in a water bath at 37° C. for 5 min. Then the cell volume was adjusted to 50 mL before centrifugation. The cell pellet thus formed was resuspended at a density of 1˜2×10⁵ cells/mL in a selection medium (HAT medium) and 0.1 mL of the cell suspension was allocated to each well of 96-well plates which were then incubated at 37° C. in a CO₂ incubator to establish a hybridoma cell population.

### 1.1.3. Selection of Hybridoma Cells Producing Monoclonal Antibodies to c-Met Protein

From the hybridoma cell population established in Reference Example 1.1.2, hybridoma cells which showed a specific response to c-Met protein were screened by ELISA using human c-Met/Fc fusion protein and human Fc protein as antigens.

Human c-Met/Fc fusion protein was seeded in an amount of 50 μL (2 μg/mL)/well to microtiter plates and allowed to adhere to the surface of each well. The antibody that remained unbound was removed by washing. For use in selecting the antibodies that do not bind c-Met but recognize Fc, human Fc protein was attached to the plate surface in the same manner.

The hybridoma cell culture obtained in Reference Example 1.1.2 was added in an amount of 50 μL to each well of the plates and incubated for 1 hour. The cells remaining unreacted were washed out with a sufficient amount of Tris-buffered saline and Tween 20 (TBST). Goat anti-mouse IgG-horseradish peroxidase (HRP) was added to the plates and incubated for 1 hour at room temperature. The plates were washed with a sufficient amount of TBST, followed by reacting the peroxidase with a substrate (OPD). Absorbance at 450 nm was measured on an ELISA reader.

Hybridoma cell lines which secrete antibodies that specifically and strongly bind to human c-Met but not human Fc were selected repeatedly. From the hybridoma cell lines obtained by repeated selection, a single clone producing a monoclonal antibody was finally separated by limiting dilution. The single clone of the hybridoma cell line producing the monoclonal antibody was deposited with the Korean Cell Line Research Foundation, an international depository authority located at Yungun-Dong, Jongno-Gu, Seoul, Korea, on Oct. 9, 2009, with Accession No. KCLRF-BP-00220 according to the Budapest Treaty (refer to Korean Patent Laid-Open Publication No. 2011-0047698).

### 1.1.4. Production and Purification of Monoclonal Antibody

The hybridoma cell line obtained in Reference Example 1.1.3 was cultured in a serum-free medium, and the monoclonal antibody (AbF46) was produced and purified from the cell culture.

First, the hybridoma cells cultured in 50 mL of a medium (DMEM) supplemented with 10% (v/v) fetal bovine serum (FBS) were centrifuged and the cell pellet was washed twice or more with 20 mL of phosphate buffered saline (PBS) to remove the FBS therefrom. Then, the cells were resuspended in 50 mL of DMEM and incubated for 3 days at 37° C. in a CO₂ incubator.

After the cells were removed by centrifugation, the supernatant was stored at 4° C. before use or immediately used for the separation and purification of the antibody. An AKTA system (GE Healthcare) equipped with an affinity column (Protein G agarose column; Pharmacia, USA) was used to purify the antibody from 50 to 300 mL of the supernatant, followed by concentration with a filter (Amicon). The antibody in PBS was stored before use in the following examples.

### 1.2. Construction of chAbF46, a Chimeric Antibody to c-Met

A mouse antibody is apt to elicit immunogenicity in humans. To solve this problem, chAbF46, a chimeric antibody, was constructed from the mouse antibody AbF46 produced in Experimental Example 1.1.4 by replacing the constant region, but not the variable region responsible for antibody specificity, with an amino sequence of the human IgG1 antibody.

In this regard, a gene was designed to include the nucleotide sequence of “EcoRI-signal sequence-VH-NheI-CH-TGA-XhoI” (SEQ ID NO: 38) for a heavy chain and the nucleotide sequence of “EcoRI-signal sequence-VL-BsiWI-CL-TGA-XhoI” (SEQ ID NO: 39) for a light chain and synthesized. Then, a DNA fragment having the heavy chain nucleotide sequence (SEQ ID NO: 38) and a DNA fragment having the light chain nucleotide sequence (SEQ ID NO: 39) were digested with EcoRI (NEB, R0101S) and XhoI (NEB, R0146S) before cloning into a vector from the pOptiVEC™-TOPO TA Cloning Kit enclosed in an OptiCHO™ Antibody Express Kit (Cat no. 12762-019, Invitrogen), and a vector from the pcDNA™ 3.3-TOPO TA Cloning Kit (Cat no. 8300-01), respectively.

Each of the constructed vectors was amplified using Qiagen Maxiprep kit (Cat no. 12662), and a transient expression was performed using Freestyle™ MAX 293 Expression System (Invitrogen). 293 F cells were used for the expression and cultured in FreeStyle™ 293 Expression Medium in a suspension culture manner. At one day before the transient expression, the cells were provided in the concentration of 5×10⁵ cells/mL, and after 24 hours, when the cell number reached to 1×10⁶ cells/mL, the transient expression was performed. A transfection was performed by a liposomal reagent method using Freestyle™ MAX reagent (Invitrogen), wherein in a 15 mL tube, the DNA was provided in the mixture ratio of 1:1 (heavy chain DNA:light chain DNA) and mixed with 2 mL of OptiPro™ SFM (Invitrogen) (A). In another 15 mL tube, 100 μL of Freestyle™ MAX reagent and 2 mL of OptiPro™ SFM were mixed (B). This was followed by mixing (A) and (B) and incubating for 15 minutes. The obtained mixture was slowly mixed with the cells provided one day before the transient expression. After completing the transfection, the cells were incubated in 130 rpm incubator for 5 days under the conditions of 37° C., 80% humidity, and 8% CO₂.

Afterwards, the cells were incubated in DMEM supplemented with 10% (v/v) FBS for 5 hours at 37° C. under a 5% CO₂ condition and then in FBS-free DMEM for 48 hours at 37° C. under a 5% CO₂ condition.

After centrifugation, the supernatant was applied to AKTA prime (GE Healthcare) to purify the antibody. In this regard, 100 mL of the supernatant was loaded at a flow rate of 5 mL/min to AKTA Prime equipped with a Protein A column (GE Healthcare, 17-0405-03), followed by elution with an IgG elution buffer (Thermo Scientific, 21004). The buffer was exchanged with PBS to purify a chimeric antibody AbF46 (hereinafter referred to as “chAbF46”).

### 1.3. Construction of Humanized Antibody huAbF46 from Chimeric Antibody chAbF46

### 1.3.1. Heavy Chain Humanization

To design two domains H1-heavy and H3-heavy, human germline genes which share the highest identity/homology with the VH gene of the mouse antibody AbF46 purified in Reference Example 1.2 were analyzed. An Ig BLAST (www.ncbi.nlm.nih.gov/igblast/) result revealed that VH3-71 has an identity/identity/homology of 83% at the amino acid level. CDR-H1, CDR-H2, and CDR-H3 of the mouse antibody AbF46 were defined according to Kabat numbering. A design was made to introduce the CDRs of the mouse antibody AbF46 into the framework of VH3-71. Hereupon, back mutations to the amino acid sequence of the mouse AbF46 were conducted at positions 30 (S→T), 48 (V→L), 73 (D→N), and 78 (T→L). Then, H1 was further mutated at positions 83 (R→K) and 84 (A→T) to finally establish. H1-heavy (SEQ ID NO: 40) and H3-heavy (SEQ ID NO: 41).

For use in designing H4-heavy, human antibody frameworks were analyzed by a BLAST search. The result revealed that the VH3 subtype, known to be most stable, is very similar in framework and sequence to the mouse antibody AbF46. CDR-H1, CDR-H2, and CDR-H3 of the mouse antibody AbF46 were defined according to Kabat numbering and introduced into the VH3 subtype to construct H4-heavy (SEQ ID NO: 42).

### 1.3.2. Light Chain Humanization

To design two domains H1-light (SEQ ID NO: 43) and H2-light (SEQ ID NO: 44), human germline genes which share the highest identity/homology with the VH gene of the mouse antibody AbF46 were analyzed. An Ig BLAST search result revealed that VK4-1 has an identity/homology of 75% at the amino acid level. CDR-L1, CDR-L2, and CDR-L3 of the mouse antibody AbF46 were defined according to Kabat numbering. A design was made to introduce the CDR of the mouse antibody AbF46 into the framework of VK4-1. Hereupon, back mutations to the amino acid sequence of the mouse AbF46 were conducted at positions 36 (Y→H), 46 (L→M), and 49 (Y→I). Only one back mutation was conducted at position 49 (Y→I) on H2-light.

To design H3-light (SEQ ID NO: 45), human germline genes which share the highest identity/homology with the VL gene of the mouse antibody AbF46 were analyzed by a search for BLAST. As a result, VK2-40 was selected. VL and VK2-40 of the mouse antibody AbF46 were found to have an identity/homology of 61% at an amino acid level. CDR-L1, CDR-L2, and CDR-L3 of the mouse antibody were defined according to Kabat numbering and introduced into the framework of VK4-1. Back mutations were conducted at positions 36 (Y→H), 46 (L→M), and 49 (Y→I) on H3-light.

For use in designing H4-light (SEQ ID NO: 46), human antibody frameworks were analyzed. A Blast search revealed that the Vk1 subtype, known to be the most stable, is very similar in framework and sequence to the mouse antibody AbF46. CDR-L1, CDR-L2, and CDR-L3 of the mouse antibody AbF46 were defined according to Kabat numbering and introduced into the Vk1 subtype. Hereupon, back mutations were conducted at positions 36 (Y→H), 46 (L→M), and 49 (Y→I) on H4-light.

Thereafter, DNA fragments having the heavy chain nucleotide sequences (H1-heavy: SEQ ID NO: 47, H3-heavy: SEQ ID NO: 48, H4-heavy: SEQ ID NO: 49) and DNA fragments having the light chain nucleotide sequences (H1-light: SEQ ID NO: 50, H2-light: SEQ ID NO: 51, H3-light: SEQ ID NO: 52, H4-light: SEQ ID NO: 53) were digested with EcoRI (NEB, R0101S) and XhoI (NEB, R0146S) before cloning into a vector from the pOptiVEC™-TOPO TA Cloning Kit enclosed in an OptiCHO™ Antibody Express Kit (Cat no. 12762-019, Invitrogen) and a vector from the pcDNA™ 3.3-TOPO TA Cloning Kit (Cat no. 8300-01), respectively, so as to construct recombinant vectors for expressing a humanized antibody.

Each of the constructed vectors was amplified using Qiagen Maxiprep kit (Cat no. 12662), and a transient expression was performed using Freestyle™ MAX 293 Expression System (Invitrogen). 293 F cells were used for the expression and cultured in FreeStyle™ 293 Expression Medium in a suspension culture manner. At one day before the transient expression, the cells were provided in the concentration of 5×10⁵ cells/mL, and after 24 hours, when the cell number reached to 1×10⁶ cells/mL, the transient expression was performed. A transfection was performed by a liposomal reagent method using Freestyle™ MAX reagent (Invitrogen), wherein in a 15 mL tube, the DNA was provided in the mixture ratio of 1:1 (heavy chain DNA:light chain DNA) and mixed with 2 mL of OptiPro™ SFM (Invitrogen) (A). In another 15 mL tube, 100 μL of Freestyle™ MAX reagent and 2 mL of OptiPro™ SFM were mixed (B). This waws followed by mixing (A) and (B) and incubating for 15 minutes. The obtained mixture was slowly mixed with the cells provided one day before the transient expression. After completing the transfection, the cells were incubated in 130 rpm incubator for 5 days under the conditions of 37° C., 80% humidity, and 8% CO₂.

After centrifugation, the supernatant was applied to AKTA prime (GE Healthcare) to purify the antibody. In this regard, 100 mL of the supernatant was loaded at a flow rate of 5 mL/min to AKTA Prime equipped with a Protein A column (GE Healthcare, 17-0405-03), followed by elution with an IgG elution buffer (Thermo Scientific, 21004). The buffer was exchanged with PBS to purify a humanized antibody AbF46 (hereinafter referred to as “huAbF46”). The humanized antibody huAbF46 used in the following examples included a combination of H4-heavy (SEQ ID NO: 42) and H4-light (SEQ ID NO: 46).

### 1.4. Construction of scFV Library of huAbF46 Antibody

For use in constructing an scFv of the huAbF46 antibody from the heavy and light chain variable regions of the huAbF46 antibody, a gene was designed to have the structure of “VH-linker-VL” for each of the heavy and the light chain variable region, with the linker including the amino acid sequence “GLGGLGGGGSGGGGSGGSSGVGS” (SEQ ID NO: 54). A polynucleotide sequence (SEQ ID NO: 55) encoding the designed scFv of huAbF46 was synthesized in Bioneer and an expression vector for the polynucleotide had the nucleotide sequence of SEQ ID NO: 56.

After expression, the product was found to exhibit specificity to c-Met.

### 1.5. Construction of Library Genes for Affinity Maturation

### 1.5.1. Selection of Target CDRs and Synthesis of Primers

The affinity maturation of huAbF46 was achieved. First, six complementary determining regions (CDRs) were defined according to Kabat numbering. The CDRs are given in Table 1, below.

TABLE 1

| CDR | Amino Acid Sequence |
|---|---|
| CDR-H1 | DYYMS |
| | (SEQ ID NO: 1) |
| CDR-H2 | FIRNKANGYTTEYSASVKG |
| | (SEQ ID NO: 2) |
| CDR-H3 | DNWFAY |
| | (SEQ ID NO: 3) |
| CDR-L1 | KSSQSLLASGNQNNYLA |
| | (SEQ ID NO: 10) |
| CDR-L2 | WASTRVS |
| | (SEQ ID NO: 11) |
| CDR-L3 | QQSYSAPLT |
| | (SEQ ID NO: 12) |

For use in the introduction of random sequences into the CDRs of the antibody, primers were designed as follows. Conventionally, N codons were utilized to introduce bases at the same ratio (25% A, 25% G, 25% C, 25% T) into desired sites of mutation. In this experiment, the introduction of random bases into the CDRs of huAbF46 was conducted in such a manner that, of the three nucleotides per codon in the wild-type polynucleotide encoding each CDR, the first and second nucleotides conserved over 85% of the entire sequence while the other three nucleotides were introduced at the same percentage (each 5%) and that the same possibility was imparted to the third nucleotide (33% G, 33% C, 33% T).

### 1.5.2. Construction of a Library of huAbF46 Antibodies and Affinity for c-Met

The construction of antibody gene libraries through the introduction of random sequences was carried out using the primers synthesized in the same manner as in Reference Example 1.5.1. Two PCR products were obtained using a polynucleotide covering the scFV of huAbF46 as a template, and were subjected to overlap extension PCR to give scFv library genes for huAbF46 antibodies in which only desired CDRs were mutated. Libraries targeting each of the six CDRs prepared from the scFV library genes were constructed.

The affinity for c-Met of each library was compared to that of the wildtype. Most libraries were lower in affinity for c-Met, compared to the wild-type. The affinity for c-Met was retained in some mutants.

1.6. Selection of Antibody with Improved Affinity from Libraries

After maturation of the affinity of the constructed libraries for c-Met, the nucleotide sequence of scFv from each clone was analyzed. The nucleotide sequences thus obtained are summarized in Table 2 and were converted into IgG forms. Four antibodies which were respectively produced from clones L3-1, L3-2, L3-3, and L3-5 were used in the subsequent experiments.

TABLE 2

| | Library | |
|---|---|---|
| Clone | constructed | CDR Sequence |
| H11-4 | CDR-H1 | PEYYMS |
| | | (SEQ ID NO: 22) |
| YC151 | CDR-H1 | PDYYMS |
| | | (SEQ ID NO: 23) |
| YC193 | CDR-H1 | SDYYMS |
| | | (SEQ ID NO: 24) |
| YC244 | CDR-H2 | RNNANGNT |
| | | (SEQ ID NO: 25) |
| YC321 | CDR-H2 | RNKVNGYT |
| | | (SEQ ID NO: 26) |
| YC354 | CDR-H3 | DNWLSY |
| | | (SEQ ID NO: 27) |
| YC374 | CDR-H3 | DNWLTY |
| | | (SEQ ID NO: 28) |
| L1-1 | CDR-L1 | KSSHSLLASGNQNNYLA |
| | | (SEQ ID NO: 29) |
| L1-3 | CDR-L1 | KSSRSLLSSGNHKNYLA |
| | | (SEQ ID NO: 30) |
| L1-4 | CDR-L1 | KSSKSLLASGNQNNYLA |
| | | (SEQ ID NO: 31) |
| L1-12 | CDR-L1 | KSSRSLLASGNQNNYLA |
| | | (SEQ ID NO: 32) |
| L1-22 | CDR-L1 | KSSHSLLASGNQNNYLA |
| | | (SEQ ID NO: 33) |
| L2-9 | CDR-L2 | WASKRVS |
| | | (SEQ ID NO: 34) |
| L2-12 | CDR-L2 | WGSTRVS |
| | | (SEQ ID NO: 35) |
| L2-16 | CDR-L2 | WGSTRVP |
| | | (SEQ ID NO: 36) |
| L3-1 | CDR-L3 | QQSYSRPYT |
| | | (SEQ ID NO: 13) |
| L3-2 | CDR-L3 | GQSYSRPLT |
| | | (SEQ ID NO: 14) |
| L3-3 | CDR-L3 | AQSYSHPFS |
| | | (SEQ ID NO: 15 |
| L3-5 | CDR-L3 | QQSYSRPFT |
| | | (SEQ ID NO: 16) |
| L3-32 | CDR-L3 | QQSYSKPFT |
| | | (SEQ ID NO: 37) |

### 1.7. Conversion of Selected Antibodies into IgG

Respective polynucleotides encoding heavy chains of the four selected antibodies were designed to have the structure of “EcoRI-signal sequence-VH-NheI-CH-XhoI” (SEQ ID NO: 38). The heavy chains of huAbF46 antibodies were used as they were because their amino acids were not changed during affinity maturation. In the case of the hinge region, however, the U6-HC7 hinge (SEQ ID NO: 57) was employed instead of the hinge of human IgG1. Genes were also designed to have the structure of “EcoRI-signal sequence-VL-BsiWI-CL-Xhol” for the light chain. Polypeptides encoding light chain variable regions of the four antibodies which were selected after the affinity maturation were synthesized in Bioneer. Then, a DNA fragment having the heavy chain nucleotide sequence (SEQ ID NO: 38) and DNA fragments having the light chain nucleotide sequences (DNA fragment comprising L3-1-derived CDR-L3: SEQ ID NO: 58, DNA fragment including L3-2-derived CDR-L3: SEQ ID NO: 59, DNA fragment comprising L3-3-derived CDR-L3: SEQ ID NO: 60, and DNA fragment comprising L3-5-derived CDR-L3: SEQ ID NO: 61) were digested with EcoRI (NEB, R0101S) and XhoI (NEB, R0146S) before cloning into a vector from the pOptiVEC™-TOPO TA Cloning Kit enclosed in an OptiCHO™ Antibody Express Kit (Cat no. 12762-019, Invitrogen) and a vector from the pcDNA™ 3.3-TOPO TA Cloning Kit (Cat no. 8300-01), respectively, so as to construct recombinant vectors for expressing affinity-matured antibodies.

Each of the constructed vectors was amplified using Qiagen Maxiprep kit (Cat no. 12662), and a transient expression was performed using Freestyle™ MAX 293 Expression System (Invitrogen). 293 F cells were used for the expression and cultured in FreeStyle™ 293 Expression Medium in a suspension culture manner. At one day before the transient expression, the cells were provided in the concentration of 5×10⁵ cells/mL, and after 24 hours, when the cell number reached to 1×10⁶ cells/mL, the transient expression was performed. A transfection was performed by a liposomal reagent method using Freestyle™ MAX reagent (Invitrogen). In a 15 mL tube, the DNA was provided in the mixture ratio of 1:1 (heavy chain DNA:light chain DNA) and mixed with 2 mL of OptiPro™ SFM (Invitrogen) (A), and in another 15 mL tube, 100 ul (microliter) of Freestyle™ MAX reagent and 2 mL of OptiPro™ SFM were mixed (B). This was followed by mixing (A) and (B) and incubating for 15 minutes. The obtained mixture was slowly mixed with the cells provided one day before the transient expression. After completing the transfection, the cells were incubated in 130 rpm incubator for 5 days under the conditions of 37° C., 80% humidity, and 8% CO₂.

After centrifugation, the supernatant was applied to AKTA prime (GE Healthcare) to purify the antibody. In this regard, 100 mL of the supernatant was loaded at a flow rate of 5 mL/min to AKTA Prime equipped with a Protein A column (GE healthcare, 17-0405-03), followed by elution with an IgG elution buffer (Thermo Scientific, 21004). The buffer was exchanged with PBS to purify four affinity-matured antibodies (hereinafter referred to as “huAbF46-H4-A1 (L3-1 origin), huAbF46-H4-A2 (L3-2 origin), huAbF46-H4-A3 (L3-3 origin), and huAbF46-H4-A5 (L3-5 origin),” respectively).

### 1.8. Construction of Constant Region- and/or Hinge Region-Substituted huAbF46-H4-A1

Among the four antibodies selected in Reference Example 1.7, huAbF46-H4-A1 was found to be the highest in affinity for c-Met and the lowest in Akt phosphorylation and c-Met degradation degree. In the antibody, the hinge region, or the constant region and the hinge region, were substituted.

The antibody huAbF46-H4-A1 (U6-HC7) was composed of a heavy chain comprising (a) the heavy chain variable region of huAbF46-H4-A1, U6-HC7 hinge, and the constant region of human IgG1 constant region, and (b) a light chain including the light chain variable region of huAbF46-H4-A1 and human kappa constant region. The antibody huAbF46-H4-A1 (IgG2 hinge) was composed of (a) a heavy chain comprising a heavy chain variable region, a human IgG2 hinge region, and a human IgG1 constant region, and (b) a light chain comprising the light chain variable region of huAbF46-H4-A1 and a human kappa constant region. The antibody huAbF46-H4-A1 (IgG2 Fc) was composed of (a) the heavy chain variable region of huAbF46-H4-A1, a human IgG2 hinge region, and a human IgG2 constant region, and (b) a light chain including the light variable region of huAbF46-H4-A1 and a human kappa constant region. Hereupon, the histidine residue at position 36 on the human kappa constant region of the light chain was changed to tyrosine in all of the three antibodies to increase antibody production.

For use in constructing the three antibodies, (a) a polynucleotide (SEQ ID NO: 63) encoding a polypeptide (SEQ ID NO: 62) composed of the heavy chain variable region of huAbF46-H4-A1, a U6-HC7 hinge region, and a human IgG1 constant region, (b) a polynucleotide (SEQ ID NO: 65) encoding a polypeptide (SEQ ID NO: 64) composed of the heavy chain variable region of huAbF46-H4-A1, a human IgG2 hinge region, and a human IgG1 region, (c) a polynucleotide (SEQ ID NO: 67) encoding a polypeptide (SEQ ID NO: 66) composed of the heavy chain variable region of huAbF46-H4-A1, a human IgG2 region, and a human IgG2 constant region, and (d) a polynucleotide (SEQ ID NO: 69) encoding a polypeptide (SEQ ID NO: 68) composed of the light chain variable region of huAbF46-H4-A1, with a tyrosine residue instead of histidine at position 36, and a human kappa constant region were synthesized in Bioneer. Then, the DNA fragments having heavy chain nucleotide sequences were inserted into a vector from the pOptiVEC™-TOPO TA Cloning Kit enclosed in an OptiCHO™ Antibody Express Kit (Cat no. 12762-019, Invitrogen) while DNA fragments having light chain nucleotide sequences were inserted into a vector from the pcDNA™ 3.3-TOPO TA Cloning Kit (Cat no. 8300-01) so as to construct vectors for expressing the antibodies.

Each of the constructed vectors was amplified using Qiagen Maxiprep kit (Cat no. 12662), and a transient expression was performed using Freestyle™ MAX 293 Expression System (Invitrogen). 293 F cells were used for the expression and cultured in FreeStyle™ 293 Expression Medium in a suspension culture manner. At one day before the transient expression, the cells were provided in the concentration of 5×10⁵ cells/mL, and after 24 hours, when the cell number reached to 1×10⁶ cells/mL, the transient expression was performed. A transfection was performed by a liposomal reagent method using Freestyle™ MAX reagent (Invitrogen), wherein in a 15 mL tube, the DNA was provided in the mixture ratio of 1:1 (heavy chain DNA:light chain DNA) and mixed with 2 mL of OptiPro™ SFM (Invitrogen) (A). In another 15 mL tube, 100 μL of Freestyle™ MAX reagent and 2 mL of OptiPro™ SFM were mixed (B). This was followed by mixing (A) and (B) and incubating for 15 minutes. The obtained mixture was slowly mixed with the cells provided one day before the transient expression. After completing the transfection, the cells were incubated in 130 rpm incubator for 5 days under the conditions of 37° C., 80% humidity, and 8% CO₂.

After centrifugation, the supernatant was applied to AKTA prime (GE Healthcare) to purify the antibody. In this regard, 100 mL of the supernatant was loaded at a flow rate of 5 mL/min to AKTA Prime equipped with a Protein A column (GE Healthcare, Cat. No. 17-0405-03), followed by elution with IgG elution buffer (Thermo Scientific, Cat. No. 21004). The buffer was exchanged with PBS to finally purify three antibodies (huAbF46-H4-A1 (U6-HC7), huAbF46-H4-A1 (IgG2 hinge), and huAbF46-H4-A1 (IgG2 Fc)). Among the three antibodies, huAbF46-H4-A1 (U6-HC7) was selected for the following examples, and referred as anti-c-Met antibody L3-1Y.

### Example 1

### Screening of Receptor Expression

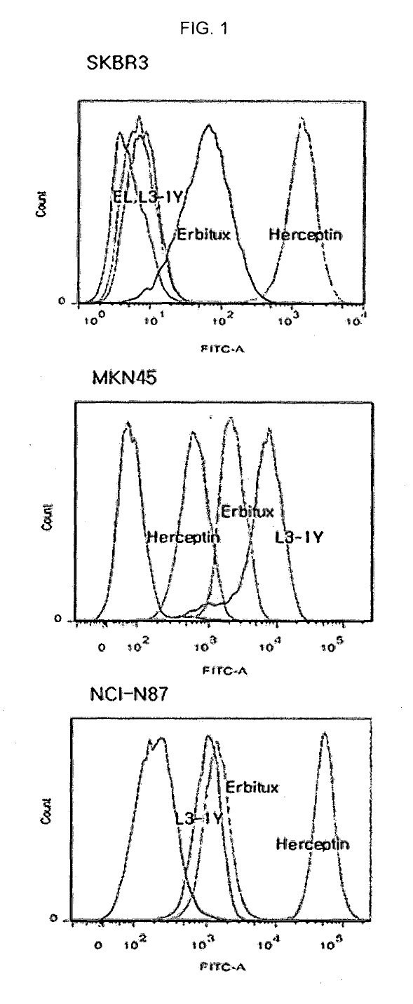

In various human cancer cell lines purchased from ATCC (SKBR3 (breast cancer cell line), MKN45 (gastric cancer cell line), NCI-N87 (gastric cancer cell line)), expressions of cMET and HER2 were examined. Specifically, 5×10⁵ cells were incubated with 1 μg/mL of a primary antibody (c-Met(L3-1Y), EGFR (Erbitux), HER2 (Herceptin)) at 4° C. for 1 hour in a FACS buffer, and then, incubated with a secondary antibody of anti-human-FITC (Jackson ImmunoResearch) at 4° C. for 30 minutes, and analyzed by FACS (FACS Canto, BD), and the results are shown in FIG. 1.

As shown in FIG. 1, particularly, high HER2 expression was observed in NCI-N87 (gastric cancer cell line), which was used in the following Examples.

### Example 2

### Confirmation of the Effect of Combination Treatment of Anti-cMet Antibody L3-1Y and Anti-HER2 Antibody Herceptin

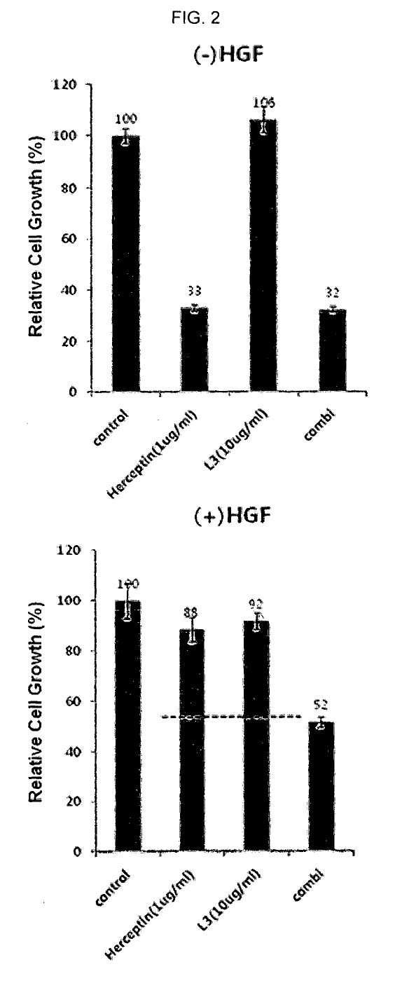

NCI-N87 cells were seeded at 5×10³ cells/well in a 96 well plate, and then, the 96 wells were treated with Herceptin and L3-1Y each alone or in combination under 5% (v/v) FBS, without HGF or with HGF(100 ng/mL) conditions (37° C., 96 hours). At 96 hours, NCI-N87 cells were measured with Cell-Titer Glo (Promega), and the results are shown in FIG. 2. The HGF used in this experiment was purchased from PANGEN.

As shown in FIG. 2, when an anti-HER2 antibody Herceptin was treated alone under conditions without HGF, an excellent inhibition effect of cell line growth was exhibited. Under conditions with HGF, acquired resistance was exhibited, and when Herceptin was treated alone, cell line growth reached 88%, thus confirming that inhibition effect of cancer cell growth was significantly decreased. However, when an anti-c-Met antibody L3-1Y and an anti-HER2 antibody Herceptin were co-treated under conditions with HGF, cell line growth was very low (as low as 52%), thus confirming synergistic effects by combination treatment of the two antibodies. Additionally, these results confirm that the limitation of Herceptin single treatment may be effectively overcome.

### Example 3

### Confirmation of the Inhibition Effect of Migration by Combination Treatment of Anti-cMet Antibody L3-1Y and Anti-HER2 Antibody Herceptin

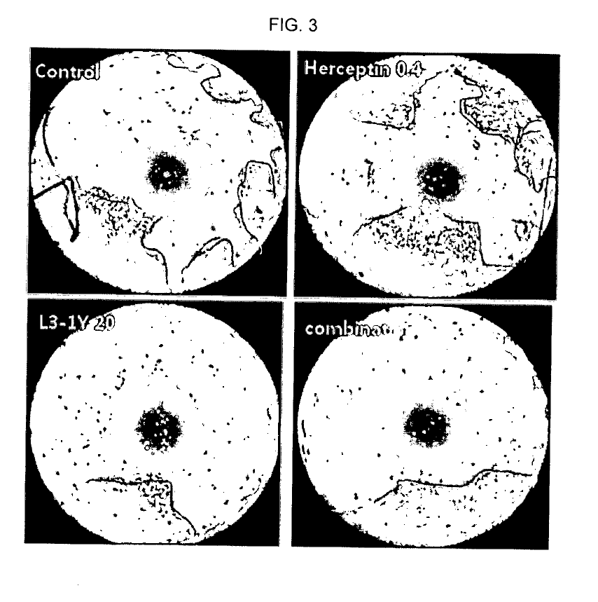

The effect of inhibiting migration by combination treatment of an anti-c-Met antibody L3-1Y and an anti-HER2 antibody Herceptin was confirmed using an ORIS™_CELL_MIGRATION Assay Kit (PLATYPUS) according to the manufacturer's instructions. Specifically, NCI-N87 cells were seeded at 1×10⁴cells/well in a 96 well plate included in the kit under conditions with HGF (100 ng/mL) (PANGEN) Then, the cells were treated with Herceptin and L3 -1 Y each alone or in combination (Herceptin 0.4 μg/mL, L3-1Y 20 μg/mL), and incubated for 72 hours. Microscope images were obtained as shown in FIG. 3.

As shown in FIG. 3, if HGF exists, an increase in cell migration was observed, and Herceptin single treatment cannot inhibit increased migration. On the contrary, combination treatment of L3-1Y and Herceptin significantly decreased cell migration. Thus, Applicants discovered that the combination treatment of an anti-c-Met antibody and an anti-HER2 antibody may inhibit cell migration while lowering cell growth as (see Example 2). Inhibition of migration may affect decreased metastasis in cancer patients.

### Example 4

### Selection of Genes Lowered by Combination Treatment of Anti c-Met Antibody and Anti-HER2 Antibody

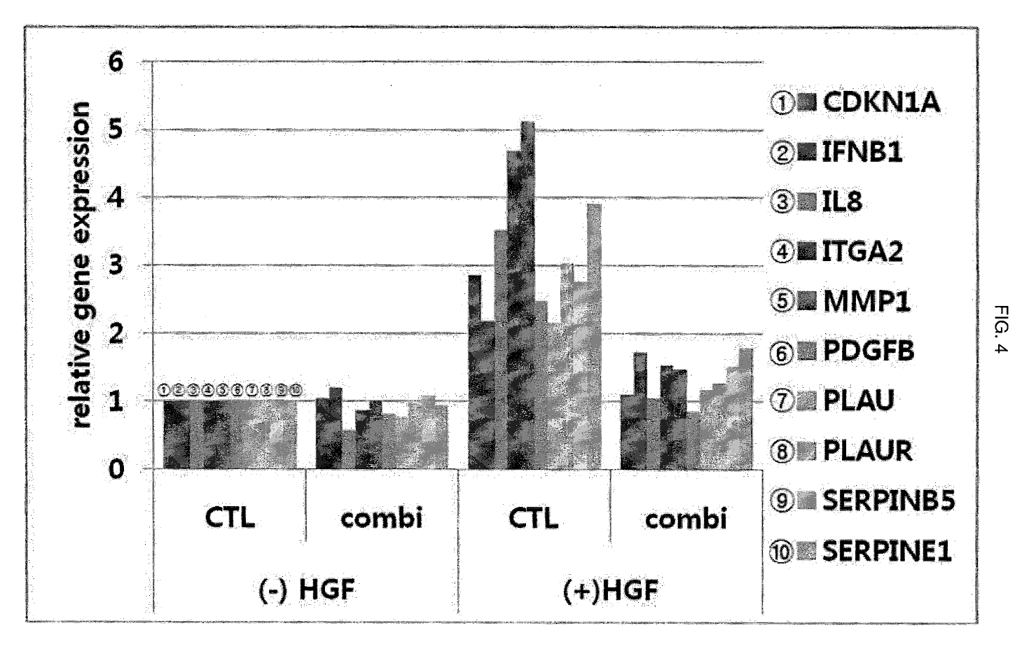

Genes whose expression may be changed by combination treatment of an anti-c-Met antibody and an anti-HER2 antibody under conditions with or without HGF were examined through microarray analysis. The results are shown in FIG. 4.

Specifically, NCI-N87 cells were seeded at 5×10⁵ cells/well in a 6 well plate. The cells were co-treated with L3-1Y and Herceptin (incubation under HGF 100 ng/mL conditions for 24 hours). Then, RNA prep was conducted using an RNeasy Kit (Qiagen) according to the manufacturer's instructions. A Cancer Pathway Finder™ PCR Array Kit and related reagents were purchased from SABioscience, and Real-Time PCR was conducted according to the manufacturer's instructions. As a PCR apparatus, LC4801 (Roche) was used.

As shown in FIG. 4, although promotion of expression of various genes was observed under conditions with HGF, significantly decreased expression of certain genes (CDKNIA, IFNB1, IL-8, and the like) was observed when L3-1Y and Herceptin were co-administered.

### Example 5

### Confirmation of the Influence of Combination Treatment of an anti c-Met antibody and Anti-HER2 Antibody on IL-8 Protein Level

Among the genes for which changes in expression were observed, those exhibiting a decrease in protein level, as well as gene level, by combination treatment were identified.

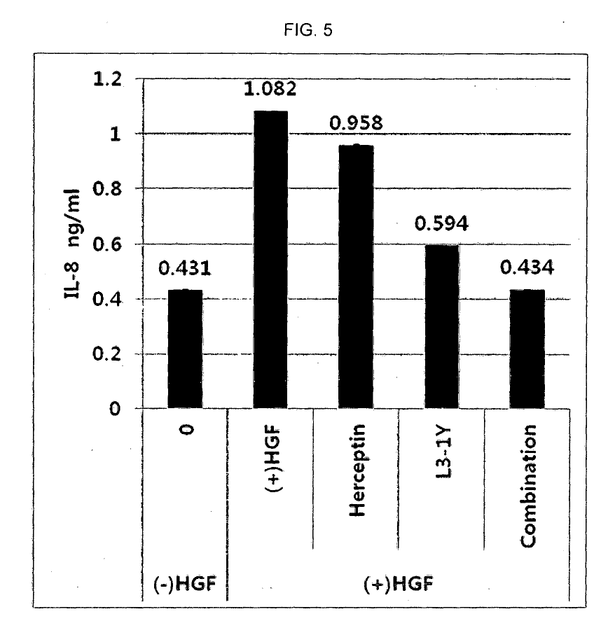

Specifically, NCI-N87 cells were seeded at 5×10⁵cell/well in a 6 well plate. The cells then were treated with L3-1Y and Herceptin each alone or in combination under HGF 100 ng/mL conditions for 72 hours. The Media soup was gathered, measured with an ELISA (IL-8 Platinum ELISA, eBioscience) kit, and shown in FIG. 5.

As shown in FIG. 5, it was determined that in the case of IL-8, the protein level significantly decreased equivalently to that observed under conditions without HGF by combination treatment of L3-1Y and Herceptin. IL-8 is a potent pro-angiogenic factor together with VEGF, and thus, it is expected that the combination treatment of an anti-c-Met antibody and an anti-HER2 antibody may achieve additional effects of decreasing angiogenesis factors.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-19. (canceled)

20. A method for prevention or treatment of a cancer or cancer metastasis, comprising co-administering (a) an anti-c-Met antibody or an antigen-binding fragment thereof and (b) an anti-HER2 antibody or an antigen-binding fragment thereof to a patient in need thereof,
wherein the anti-c-Met antibody or the antigen-binding fragment thereof specifically binds to an epitope comprising 5 or more contiguous amino acids within the SEMA domain of c-Met protein,
thereby preventing or treating cancer or cancer metastasis in the patient.

21. The method of claim 20, wherein the anti-c-Met antibody or the antigen-binding fragment thereof and the anti-HER2 antibody or the antigen-binding fragment thereof are administered simultaneously or sequentially in any order.

22. The method of claim 20, wherein the anti-HER2 antibody is selected from the group consisting of trastuzumab, pertuzumab, trastuzumab emtansine, and a combination thereof.

23. The method of claim 20, wherein the anti c-Met antibody or the antigen-binding fragment thereof specifically binds to an epitope comprising 5 to 19 contiguous amino acids of SEQ ID NO: 71, and wherein the epitope comprises the amino acid sequence of SEQ ID NO: 73.

24. The method of claim 20, wherein the anti c-Met antibody or the antigen-binding fragment comprises:
(a) a heavy chain variable region comprising at least one heavy chain complementarity determining region (CDR) selected from the group consisting of (i) a CDR-H1 comprising the amino acid sequence of SEQ ID NO: 4; (ii) a CDR-H2 comprising the amino acid sequence of SEQ ID NO: 5, the amino acid sequence of SEQ ID NO: 2, or an amino acid sequence comprising 8-19 consecutive amino acids of the amino acid sequence of SEQ ID NO: 2, wherein the 8-19 consecutive amino acids comprise amino acid residues from the 3^{rd} to 10^{th} positions of the amino acid sequence of SEQ ID NO: 2; and (iii) a CDR-H3 comprising the amino acid sequence of SEQ ID NO: 6, the amino acid sequence of SEQ ID NO: 85, or an amino acid sequence comprising 6-13 consecutive amino acids of the amino acid sequence of SEQ ID NO: 85, wherein the 6-13 consecutive amino acids comprise amino acid residues from the 1^{st} to 6^{th} positions of the amino acid sequence of SEQ ID NO: 85; and
(b) a light chain variable region comprising at least one light chain complementarity determining region (CDR) selected from the group consisting of (i) a CDR-L1 comprising the amino acid sequence of SEQ ID NO: 7, (ii) a CDR-L2 comprising the amino acid sequence of SEQ ID NO: 8, and (iii) a CDR-L3 comprising the amino acid sequence of SEQ ID NO: 9, the amino acid sequence of SEQ ID NO: 15, the amino acid sequence of SEQ ID NO: 86, or an amino acid sequence comprising 9-17 consecutive amino acids of the amino acid sequence of SEQ ID NO: 89, wherein the 9-17 consecutive amino acids comprise amino acid residues from the 1^{st} to 9^{th} positions of the amino acid sequence of the SEQ ID NO: 89.

25. The method of claim 24, wherein
the CDR-H1 comprises the amino acid sequence of SEQ ID NO: 1, SEQ ID NO: 22, SEQ ID NO: 23, or SEQ ID NO: 24,
the CDR-H2 comprises the amino acid sequence of SEQ ID NO: 2, SEQ ID NO: 25, or SEQ ID NO: 26,
the CDR-H3 comprises the amino acid sequence of SEQ ID NO: 3, SEQ ID NO: 27, SEQ ID NO: 28, or SEQ ID NO: 85,
the CDR-L1 comprises the amino acid sequence of SEQ ID NO: 10, SEQ ID NO: 29, SEQ ID NO: 30, SEQ ID NO: 31, SEQ ID NO: 32, SEQ ID NO: 33, or SEQ ID NO: 106,
the CDR-L2 comprises the amino acid sequence of SEQ ID NO: 11, SEQ ID NO: 34, SEQ ID NO: 35, or SEQ ID NO: 36, and
the CDR-L3 comprises the amino acid sequence of SEQ ID NO: 12, SEQ ID NO: 13, SEQ ID NO: 14, SEQ ID NO: 15, SEQ ID NO: 16, SEQ ID NO: 37, SEQ ID NO: 86, or SEQ ID NO: 89.

26. The method of claim 24, wherein
(a) the heavy chain variable region comprises the amino acid sequence of SEQ ID NO: 17, SEQ ID NO: 74, SEQ ID NO: 87, SEQ ID NO: 90, SEQ ID NO: 91, SEQ ID NO: 92, SEQ ID NO: 93, or SEQ ID NO: 94, and
(b) the light chain variable region comprises the amino acid sequence of SEQ ID NO: 109 SEQ ID NO: 18, SEQ ID NO: 19, SEQ ID NO: 20, SEQ ID NO: 21, SEQ ID NO: 75, SEQ ID NO: 88, SEQ ID NO: 95, SEQ ID NO: 96, SEQ ID NO: 97, SEQ ID NO: 98, SEQ ID NO: 99, or SEQ ID NO: 107.

27. The method of claim 24, wherein the anti-c-Met antibody comprises:
(a) a heavy chain comprising (i) the amino acid sequence of SEQ ID NO: 62, (ii) the amino acid sequence from the 18^{th} to 462^{nd} positions of the amino acid sequence of SEQ ID NO: 62, (iii) the amino acid sequence of SEQ ID NO: 64, (iv) the amino acid sequence from the 18^{th} to 461^{st} positions of the amino acid sequence of SEQ ID NO: 64, (v) the amino acid sequence of SEQ ID NO: 66, or (vi) the amino acid sequence from the 18^{th} to 460^{th} positions of the amino acid sequence of SEQ ID NO: 66; and
(b) a light chain comprising (i) the amino acid sequence of SEQ ID NO: 68, (ii) the amino acid sequence from the 21^{st} to 240^{th} positions of the amino acid sequence of SEQ ID NO: 68, (iii) the amino acid sequence of SEQ ID NO: 70, (iv) the amino acid sequence from the 21^{st} to 240^{th} positions of the amino acid sequence of SEQ ID NO: 70, or (v) the amino acid sequence of SEQ ID NO: 108.

28. The method of claim 24, wherein the anti-c-Met antibody comprises a light chain complementarity determining region comprising the amino acid sequence of SEQ ID NO: 106, a light chain variable region comprising the amino acid sequence of SEQ ID NO: 107, or a light chain comprising the amino acid sequence of SEQ ID NO: 108.

29. The method of claim 20, wherein the anti-c-Met antibody is a monoclonal antibody.

30. The method of claim 20, wherein the anti-c-Met antibody is a mouse originated antibody, a mouse-human chimeric antibody, a humanized antibody, or a human antibody.

31. The method of claim 20, wherein the antigen-binding fragment is selected from the group consisting of scFv, (scFv)₂, Fab, Fab′, and F(ab′)₂ of the anti-c-Met antibody.

32. A method for inhibition of angiogenesis, comprising co-administering (a) an anti-c-Met antibody or an antigen-binding fragment thereof and (b) an anti-HER2 antibody or an antigen-binding fragment thereof to a patient in need thereof,
wherein the anti-c-Met antibody or the antigen-binding fragment thereof specifically binds to an epitope comprising 5 or more contiguous amino acids within the SEMA domain of c-Met protein,
thereby inhibiting angiogenesis in the patient.

33. The method of claim 32, wherein the anti-c-Met antibody or the antigen-binding fragment thereof and the anti-HER2 antibody or the antigen-binding fragment thereof are administered simultaneously or sequentially in any order.

34. The method of claim 32, wherein anti-HER2 antibody is selected from the group consisting of trastuzumab, pertuzumab, trastuzumab emtansine, and a combination thereof.

35. The method of claim 32, wherein the anti c-Met antibody or the antigen-binding fragment thereof specifically binds to an epitope comprising 5 to 19 contiguous amino acids of the amino acid sequence of SEQ ID NO: 71, and wherein the epitope comprises the amino acid sequence of the amino acid sequence of SEQ ID NO: 73.

36. The method of claim 32, wherein the anti c-Met antibody or the antigen-binding fragment comprises:
(a) a heavy chain variable region comprising at least one heavy chain complementarity determining region (CDR) selected from the group consisting of (i) a CDR-H1 comprising the amino acid sequence of SEQ ID NO: 4; (ii) a CDR-H2 comprising the amino acid sequence of SEQ ID NO: 5, the amino acid sequence of SEQ ID NO: 2, or an amino acid sequence comprising 8-19 consecutive amino acids of the amino acid sequence of SEQ ID NO: 2, wherein the 8-19 contiguous amino acids comprise amino acid residues from the 3^{rd} to 10^{th} positions of the amino acid sequence of SEQ ID NO: 2; and (iii) a CDR-H3 comprising the amino acid sequence of SEQ ID NO: 6, the amino acid sequence of SEQ ID NO: 85, or an amino acid sequence comprising 6-13 consecutive amino acids of the amino acid sequence of SEQ ID NO: 85, wherein the 6-13 consecutive amion acids comprises amino acid residues from the 1^{st} to 6^{th} positions of the amino acid sequence of SEQ ID NO: 85; and
(b) a light chain variable region comprising at least one light chain CDR selected from the group consisting of (i) a CDR-L1 comprising the amino acid sequence of SEQ ID NO: 7, (ii) a CDR-L2 comprising the amino acid sequence of SEQ ID NO: 8, and (iii) a CDR-L3 comprising the amino acid sequence of SEQ ID NO: 9, the amino acid sequence of SEQ ID NO: 15, the amino acid sequence of SEQ ID NO: 86, or an amino acid sequence comprising 9-17 consecutive amino acids of the amino acid sequence of SEQ ID NO: 89, wherein the 9-17 consecutive amino acids comprise amino acid residues from the 1^{st} to 9^{th} positions of the amino acid sequence of SEQ ID NO: 89.
